(19) United States
(12) Patent Application Publication
(10) Pub. No.: US 2025/0195412 A1
(43) Pub. Date: Jun. 19, 2025

(54) PHARMACEUTICAL FORMULATIONS OF R-KETAMINE

(71) Applicant: Perception Neuroscience, Inc., New York, NY (US)

(72) Inventors: Mouhannad JUMAA, San Jose, CA (US); Terence Alfred KELLY, Ridgefield, CT (US)

(21) Appl. No.: 18/845,699

(22) PCT Filed: Mar. 13, 2023
(86) PCT No.: PCT/US2023/064236

§ 371 Date: Sep. 10, 2024

Related U.S. Application Data
(60) Provisional application No. 63/319,504, filed on Mar. 14, 2022.

(51) Int. Cl. A61K 9/00; A61K 9/08; A61K 31/135; A61K 47/02; A61K 47/12

(52) U.S. Cl. CPC A61K 9/0019; A61K 9/08; A61K 31/135; A61K 47/02; A61K 47/12

(57) ABSTRACT

The present disclosure is directed to R-ketamine formulations for subcutaneous administration. In some embodiments, the formulations described herein are optionally substantially free of S-ketamine and pharmaceutically acceptable salts thereof. In some embodiments, the formulations described herein comprise R-ketamine at a concentration between about 90 mg/mL and about 110 mg/mL at a suitable pH.

### CROSS REFERENCE TO RELATED APPLICATIONS

This application claims priority to, and benefit of, U.S. Provisional Application No. 63/319,504, filed on Mar. 14, 2022, the contents of which are incorporated by reference in their entirety herein.

### BACKGROUND

R-ketamine has been shown to be active in the treatment of various neurological conditions and in alleviating corresponding symptoms. Optimizing the effects of administering R-ketamine requires the development of new, stable and useful formulations. The present disclosure helps solve the clinical need for the administration of R-ketamine using a composition formulated for subcutaneous administration.

### SUMMARY

In one aspect, the present disclosure is directed to a formulation for subcutaneous administration comprising R-ketamine or a pharmaceutically acceptable salt thereof.

In some embodiments, the formulation for subcutaneous administration comprising R-ketamine, or a pharmaceutically acceptable salt thereof, is substantially free of S-ketamine and/or a pharmaceutically acceptable salt thereof.

In some embodiments, the formulation for subcutaneous administration comprising R-ketamine comprises R-ketamine at a concentration between about 90 mg/mL and about 110 mg/mL.

In some embodiments, the formulation for subcutaneous administration comprising R-ketamine comprises R-ketamine at a concentration between about 95 mg/mL and about 110 mg/mL.

In some embodiments, the formulation for subcutaneous administration comprising R-ketamine comprises R-ketamine at a concentration between about 90 mg/mL and about 105 mg/mL.

In some embodiments, the formulation for subcutaneous administration comprising R-ketamine comprises R-ketamine at a concentration between about 95 mg/mL and about 105 mg/mL.

In some embodiments, the formulation for subcutaneous administration comprising R-ketamine comprises R-ketamine at a concentration of about 90 mg/mL, about 95 mg/mL, about 100 mg/mL, about 105 mg/mL or about 110 mg/mL.

In some embodiments, the pH of the formulation for subcutaneous administration comprising R-ketamine is about 6.0 or less.

In some embodiments, the pH of the formulation for subcutaneous administration comprising R-ketamine is about 5.8 or less.

In some embodiments, the pH of the formulation for subcutaneous administration comprising R-ketamine is between about 5.70 and about 5.80.

In some embodiments, the pH of the formulation for subcutaneous administration comprising R-ketamine is below a pH of about 5.75.

In some embodiments, the pH of the formulation for subcutaneous administration comprising R-ketamine is at a pH of about 5.8.

In some embodiments, the pH of the formulation for subcutaneous administration comprising R-ketamine is at a pH of about 5.75.

In some embodiments, the formulation for subcutaneous administration comprising R-ketamine further comprises a buffer.

In some embodiments, the formulation for subcutaneous administration comprising R-ketamine further comprises a buffer selected from one or more of a succinate buffer, tartrate buffer, maleate buffer, a fumarate buffer, a citrate buffer, and an acetate buffer.

In some embodiments, the formulation for subcutaneous administration comprises a pharmaceutically acceptable salt of R-ketamine.

In one aspect, the present disclosure is directed to an R-ketamine formulation for subcutaneous administration comprising:
a) R-ketamine between about 90 mg/mL and about 110 mg/mL; and
b) a buffering agent;
wherein the pH of the formulation is 5.8 or less, and wherein the formulation is substantially free of S-ketamine.

In some embodiments, is the pH of the formulation for subcutaneous administration comprising R-ketamine is at a pH of about 5.7 or less.

In some embodiments, is the pH of the formulation for subcutaneous administration comprising R-ketamine is at a pH of about 5.7.

In some embodiments, is the pH of the formulation for subcutaneous administration comprising R-ketamine is at a pH of about 5.6 or less.

In some embodiments, is the pH of the formulation for subcutaneous administration comprising R-ketamine is at a pH of about 5.6.

In some embodiments, is the pH of the formulation for subcutaneous administration comprising R-ketamine is at a pH of about 5.5.

In some embodiments, the formulation for subcutaneous administration comprising R-ketamine comprises a buffering agent selected from one or more of a succinate buffer, tartrate buffer, maleate buffer, a fumarate buffer, a citrate buffer and an acetate buffer.

In some embodiments, the formulation for subcutaneous administration comprising R-ketamine comprises a buffering agent, wherein the buffering agent is a succinate buffer.

In some embodiments, the formulation for subcutaneous administration comprising R-ketamine comprises a buffering agent, wherein the buffering agent is a tartrate buffer.

In some embodiments, the formulation for subcutaneous administration comprising R-ketamine comprises a buffering agent, wherein the buffering agent is a maleate buffer.

In some embodiments, the formulation for subcutaneous administration comprising R-ketamine comprises a buffering agent, wherein the buffering agent is a fumarate buffer.

In some embodiments, the formulation for subcutaneous administration comprising R-ketamine comprises a buffering agent, wherein the buffering agent is a citrate buffer.

In some embodiments, the formulation for subcutaneous administration comprising R-ketamine comprises a buffering agent, wherein the buffering agent is an acetate buffer.

In one aspect, the present disclosure is directed to a process for preparing a formulation for subcutaneous administration comprising R-ketamine.

In some embodiments, the process for preparing a formulation for subcutaneous administration comprising R-ketamine comprises adjusting the pH using a dilute acid.

In some embodiments, the process for preparing a formulation for subcutaneous administration comprising R-ketamine comprises adjusting the pH using an acidic solution with concentration less than 1N.

In some embodiments, the process for preparing a formulation for subcutaneous administration comprising R-ketamine comprises adjusting the pH using a dilute HCl solution.

In some embodiments, the process for preparing a formulation for subcutaneous administration comprising R-ketamine comprises adjusting the pH using a dilute base.

In some embodiments, the process for preparing a formulation for subcutaneous administration comprising R-ketamine comprises adjusting the pH using a basic solution with concentration less than 1N.

In some embodiments, the process for preparing a formulation for subcutaneous administration comprising R-ketamine comprises adjusting the pH using either a dilute KOH solution or a dilute NaOH solution.

In some embodiments, the process for preparing a formulation for subcutaneous administration comprising R-ketamine comprises adjusting the pH using a dilute NaOH solution.

In some embodiments, the process for preparing a formulation for subcutaneous administration comprising R-ketamine comprises adjusting the pH using a dilute KOH solution.

In some embodiments, the process for preparing a formulation for subcutaneous administration comprising R-ketamine comprises adjusting the pH using a basic solution less than 1N.

In some embodiments, the process for preparing a formulation for subcutaneous administration comprising R-ketamine comprises adjusting the pH using either a KOH solution with a concentration of 1N or less or a NaOH solution with a concentration of 1N or less.

In some embodiments, the process for preparing a formulation for subcutaneous administration comprising R-ketamine comprises adjusting the pH using a 1N NaOH solution.

In some embodiments, the process for preparing a formulation for subcutaneous administration comprising R-ketamine comprises adjusting the pH using a 1N KOH solution.

In some embodiments, the process for preparing a formulation for subcutaneous administration comprising R-ketamine comprises adjusting the pH using a 0.5N NaOH solution.

In some embodiments, the process for preparing a formulation for subcutaneous administration comprising R-ketamine comprises adjusting the pH using a 0.5N KOH solution.

In some embodiments, the process for preparing a formulation for subcutaneous administration comprising R-ketamine comprises adjusting the pH using a 0.15N NaOH solution.

In some embodiments, the process for preparing a formulation for subcutaneous administration comprising R-ketamine comprises adjusting the pH using a 0.15N KOH solution.

In some embodiments, the process discussed herein for preparing a formulation for subcutaneous administration comprising R-ketamine facilitates increased solubility of the R-ketamine. In some embodiments, the pH is adjusted slowly. In some embodiments, the pH is adjusted slowly with a dilute acid or a dilute base. In some embodiments, the pH is adjusted in a way that minimizes localized fluctuations of pH. In some embodiments, the pH is adjusted such that micro-changes in pH in the solution is minimized. In some embodiments, the pH is adjusted slowly using a dilute acid or a dilute base such that micro-changes in pH in the solution is minimized. In some embodiments, the pH is adjusted slowly using a dilute acid or a dilute base such that micro-changes in pH in the solution is minimized which improves solubility of the R-ketamine in the formulation.

In some embodiments, the process discussed herein for preparing a formulation for subcutaneous administration comprising R-ketamine facilitates increased solubility of the R-ketamine at a pH of between about 5.6 and about 5.8.

In some embodiments, the process discussed herein for preparing a formulation for subcutaneous administration comprising R-ketamine facilitates increased solubility of the R-ketamine at a pH of about 5.8.

In some embodiments, the process discussed herein for preparing a formulation for subcutaneous administration comprising R-ketamine facilitates increased solubility of the R-ketamine at a pH of about 5.75.

In some embodiments, the process discussed herein for preparing a formulation for subcutaneous administration comprising R-ketamine facilitates increased solubility of the R-ketamine at a pH of about 5.7.

In some embodiments, the process discussed herein for preparing a formulation for subcutaneous administration comprising R-ketamine facilitates increased solubility of the R-ketamine at a pH of about 5.6.

In one aspect, the present disclosure is directed to an injection device comprising a needle suitable for subcutaneous administration, wherein the device further comprises an R-ketamine formulation for subcutaneous administration.

In one aspect, the present disclosure is directed to an injection device comprising a needle suitable for subcutaneous administration, wherein the device further comprises an R-ketamine formulation for subcutaneous administration wherein the formulation is substantially free of S-ketamine.

In one aspect, the present disclosure is directed to an injection device comprising a needle suitable for subcutaneous administration, wherein the device further comprises an R-ketamine formulation for subcutaneous administration comprising:
a) R-ketamine between about 90 mg/mL and about 110 mg/mL; and
b) a buffering agent;
wherein the pH of the formulation is 5.8 or less, and wherein the formulation is substantially free of S-ketamine.

In one aspect, the present disclosure is directed to a kit comprising an R-ketamine formulation for subcutaneous administration and instructions for use thereof.

In one aspect, the present disclosure is directed to a method of treating a patient in need thereof comprising administering the R-ketamine formulation discussed herein.

In one aspect, the present disclosure is directed to a method of treating a patient in need thereof comprising administering the R-ketamine formulation via subcutaneous administration.

In one aspect, the present disclosure is directed to the use of an R-ketamine formulation discussed herein for the treatment of a neurological condition.

### BRIEF DESCRIPTION OF THE DRAWINGS

FIG. 1 shows the Henderson-Hasselbalch representation of R-ketamine across a range of pH. The text under the bracket at bottom reads “free base concentration follows Henderson-Hasselbach,” while the text in the upper left reads “Prevalent Arketamine species as a function of pH.”

FIG. 2 depicts the concentration (mg/mL) of R-ketamine and the pH value of the final solution in 50% of propylene glycol which showed an increase in R-ketamine concentration to 46.5 mg/mL compared to ˜20 mg/mL at pH 6.

FIG. 3 depicts the precipitation of 60 mg/mL R-ketamine in phosphate buffer (pH 5.8) after 2 weeks storage.

FIG. 4 shows the solubility of R-ketamine in the pH range of 5.5-5.7.

FIG. 5 shows the four-week stability of various concentrations of R-ketamine at pH 5.5-5.7.

FIG. 6 shows the impurities of R-ketamine in water for injection (WFI) after 4-week storage at room temperature (RT) at pH 5.5-5.7.

FIG. 7 shows the solubility of R-ketamine in the pH range of 5.5-5.7.

FIG. 8 shows the four-week stability of various concentrations of R-ketamine at pH 5.5-5.7.

FIG. 9 shows impurities of maleic acid buffered R-ketamine after 4-week storage at room temperature (RT) at pH 5.5-5.7.

### DETAILED DESCRIPTION

All publications, patents, and patent applications mentioned in this specification are herein incorporated by reference to the same extent as if each individual publication, patent, or patent application was specifically and individually indicated to be incorporated by reference.

As used herein, “R-ketamine” refers to the R-(−) enantiomer of ketamine (R-ketamine IUPAC name: (2R)-2-(2-chlorophenyl)-2-(methylamino)cyclohexan-1-one).

As used herein, “about” or “approximately,” when used in connection with a numerical variable, generally refers to the value of the variable and to all values of the variable that are within the experimental error (e.g., within the 95% confidence interval for the mean) or within plus or minus 10% of the indicated numerical value, whichever is greater.

As used herein, the term “a” or “an” refers to one or more of that entity. In addition, reference to “an agent” by the indefinite article “a” or “an” does not necessarily exclude the possibility that more than one of the agents are present.

As used herein, the verb “comprise” as is used in this description and in the claims and its conjugations are used in its non-limiting sense to mean that items following the word are included, but items not specifically mentioned are not excluded. The present disclosure may suitably “comprise”, “consist of”, or “consist essentially of”, the steps, elements, and/or reagents described in the claims.

As used herein, the term “subject” is used interchangeably with the term “patient” and preferably refers to a human patient. In some embodiments, the subject can be any animal, including non-human mammals, such as mice, rats, other rodents, rabbits, dogs, cats, swine, cattle, sheep, horses, or primates.

As used herein, the terms “formulation” and “composition” are used interchangeably, except where otherwise clearly intended to have different meanings.

As used herein, the term “dosage form” refers to a parenteral composition (e.g. a subcutaneous administration) in a portion that delivers a single dose, in a single administration, to a subject. The term “an effective amount” is interchangeable with “therapeutically effective dose,” or “therapeutically effective amount,” and refers to an amount sufficient to produce the desired effect. An effective amount is sufficient to cause an improvement in a condition of the subject.

As used herein, the term “parenteral or injectable” refers to administration of a drug by injection under one or more layer of skin or mucous membrane, and can include, for example, subcutaneous, intravenous, intraperitoneal or intramuscular injection.

As used herein, the term “subcutaneous administration” as used herein refers to any type of administration method used to deliver medication into the layer of fat between the skin and the muscle, including but not limited to a patch or a needle, among others. This type of administration can be given by a healthcare professional, or it can be self-injected.

As used herein, the term “subcutaneous injection” as used herein refers to a shot that delivers medication into the layer of fat between the skin and the muscle. This type of injection usually involves a needle and can be given by a healthcare professional, or it can be self-injected. In some embodiments, the formulation can be self-injected at home.

As used herein, “treating”, “treatment”, and the like, describe the administration of a pharmaceutical composition of the invention to a subject or patient for the purpose of combating a disease, condition, or disorder, which includes decreasing, mitigating or eliminating one or more symptoms or complications of the disease, condition or disorder, or decreasing, mitigating, or eliminating the disease, condition or disorder.

As used herein, “prevent”, “preventing” and the like describe stopping the onset of the disease, condition or disorder, or one or more symptoms or complications thereof.

### Formulations of the Present Disclosure

In some embodiments, the formulations can include prefilled syringes, vials, powder for infusion for reconstitution, concentrate for infusion to be diluted before delivery (ready to dilute) or solutions (ready to use).

In some embodiments, any of the salts or salt forms described in International Application No. PCT/US2022/046412 are contemplated in each aspect and/or embodiment as described herein. International Application No. PCT/US2022/046412 is hereby incorporated by reference in its entirety.

In some embodiments, formulations can be aqueous isotonic solutions or suspensions.

For subcutaneous use, a sterile solution may be employed. In some embodiments, the total concentration of the solute(s) should be controlled to render the formulation isotonic.

In some embodiments, the formulation for subcutaneous injection comprising R-ketamine comprises R-ketamine at a concentration between about 60 mg/mL and about 150 mg/mL.

In some embodiments, the formulation for subcutaneous injection comprising R-ketamine comprises R-ketamine at a concentration between about 70 mg/mL and about 150 mg/mL.

In some embodiments, the formulation for subcutaneous injection comprising R-ketamine comprises R-ketamine at a concentration between about 80 mg/mL and about 150 mg/mL.

In some embodiments, the formulation for subcutaneous injection comprising R-ketamine comprises R-ketamine at a concentration between about 90 mg/mL and about 150 mg/mL.

In some embodiments, the formulation for subcutaneous injection comprising R-ketamine comprises R-ketamine at a concentration between about 100 mg/mL and about 150 mg/mL.

In some embodiments, the formulation for subcutaneous injection comprising R-ketamine comprises R-ketamine at a concentration between about 110 mg/mL and about 150 mg/mL.

In some embodiments, the formulation for subcutaneous injection comprising R-ketamine comprises R-ketamine at a concentration between about 120 mg/mL and about 150 mg/mL.

In some embodiments, the formulation for subcutaneous injection comprising R-ketamine comprises R-ketamine at a concentration between about 130 mg/mL and about 150 mg/mL.

In some embodiments, the formulation for subcutaneous injection comprising R-ketamine comprises R-ketamine at a concentration between about 140 mg/mL and about 150 mg/mL.

In some embodiments, the formulation for subcutaneous injection comprising R-ketamine comprises R-ketamine at a concentration between about 60 mg/mL and about 140 mg/mL.

In some embodiments, the formulation for subcutaneous injection comprising R-ketamine comprises R-ketamine at a concentration between about 70 mg/mL and about 140 mg/mL.

In some embodiments, the formulation for subcutaneous injection comprising R-ketamine comprises R-ketamine at a concentration between about 80 mg/mL and about 140 mg/mL.

In some embodiments, the formulation for subcutaneous injection comprising R-ketamine comprises R-ketamine at a concentration between about 90 mg/mL and about 140 mg/mL.

In some embodiments, the formulation for subcutaneous injection comprising R-ketamine comprises R-ketamine at a concentration between about 100 mg/mL and about 140 mg/mL.

In some embodiments, the formulation for subcutaneous injection comprising R-ketamine comprises R-ketamine at a concentration between about 110 mg/mL and about 140 mg/mL.

In some embodiments, the formulation for subcutaneous injection comprising R-ketamine comprises R-ketamine at a concentration between about 120 mg/mL and about 140 mg/mL.

In some embodiments, the formulation for subcutaneous injection comprising R-ketamine comprises R-ketamine at a concentration between about 130 mg/mL and about 140 mg/mL.

In some embodiments, the formulation for subcutaneous injection comprising R-ketamine comprises R-ketamine at a concentration between about 60 mg/mL and about 130 mg/mL.

In some embodiments, the formulation for subcutaneous injection comprising R-ketamine comprises R-ketamine at a concentration between about 70 mg/mL and about 130 mg/mL.

In some embodiments, the formulation for subcutaneous injection comprising R-ketamine comprises R-ketamine at a concentration between about 80 mg/mL and about 130 mg/mL.

In some embodiments, the formulation for subcutaneous injection comprising R-ketamine comprises R-ketamine at a concentration between about 90 mg/mL and about 130 mg/mL.

In some embodiments, the formulation for subcutaneous injection comprising R-ketamine comprises R-ketamine at a concentration between about 100 mg/mL and about 130 mg/mL.

In some embodiments, the formulation for subcutaneous injection comprising R-ketamine comprises R-ketamine at a concentration between about 110 mg/mL and about 130 mg/mL.

In some embodiments, the formulation for subcutaneous injection comprising R-ketamine comprises R-ketamine at a concentration between about 120 mg/mL and about 130 mg/mL.

In some embodiments, the formulation for subcutaneous injection comprising R-ketamine comprises R-ketamine at a concentration between about 60 mg/mL and about 120 mg/mL.

In some embodiments, the formulation for subcutaneous injection comprising R-ketamine comprises R-ketamine at a concentration between about 70 mg/mL and about 120 mg/mL.

In some embodiments, the formulation for subcutaneous injection comprising R-ketamine comprises R-ketamine at a concentration between about 80 mg/mL and about 120 mg/mL.

In some embodiments, the formulation for subcutaneous injection comprising R-ketamine comprises R-ketamine at a concentration between about 90 mg/mL and about 120 mg/mL.

In some embodiments, the formulation for subcutaneous injection comprising R-ketamine comprises R-ketamine at a concentration between about 100 mg/mL and about 120 mg/mL.

In some embodiments, the formulation for subcutaneous injection comprising R-ketamine comprises R-ketamine at a concentration between about 110 mg/mL and about 120 mg/mL.

In some embodiments, the formulation for subcutaneous injection comprising R-ketamine comprises R-ketamine at a concentration between about 60 mg/mL and about 110 mg/mL.

In some embodiments, the formulation for subcutaneous injection comprising R-ketamine comprises R-ketamine at a concentration between about 70 mg/mL and about 110 mg/mL.

In some embodiments, the formulation for subcutaneous injection comprising R-ketamine comprises R-ketamine at a concentration between about 80 mg/mL and about 110 mg/mL.

In some embodiments, the formulation for subcutaneous injection comprising R-ketamine comprises R-ketamine at a concentration between about 90 mg/mL and about 110 mg/mL.

In some embodiments, the formulation for subcutaneous injection comprising R-ketamine comprises R-ketamine at a concentration between about 100 mg/mL and about 110 mg/mL.

In some embodiments, the formulation for subcutaneous injection comprising R-ketamine comprises R-ketamine at a concentration between about 60 mg/mL and about 100 mg/mL.

In some embodiments, the formulation for subcutaneous injection comprising R-ketamine comprises R-ketamine at a concentration between about 70 mg/mL and about 100 mg/mL.

In some embodiments, the formulation for subcutaneous injection comprising R-ketamine comprises R-ketamine at a concentration between about 80 mg/mL and about 100 mg/mL.

In some embodiments, the formulation for subcutaneous injection comprising R-ketamine comprises R-ketamine at a concentration between about 90 mg/mL and about 100 mg/mL.

In some embodiments, the formulation for subcutaneous injection comprising R-ketamine comprises R-ketamine at a concentration between about 60 mg/mL and about 90 mg/mL.

In some embodiments, the formulation for subcutaneous injection comprising R-ketamine comprises R-ketamine at a concentration between about 70 mg/mL and about 90 mg/mL.

In some embodiments, the formulation for subcutaneous injection comprising R-ketamine comprises R-ketamine at a concentration between about 80 mg/mL and about 90 mg/mL.

In some embodiments, the formulation for subcutaneous injection comprising R-ketamine comprises R-ketamine at a concentration between about 60 mg/mL and about 80 mg/mL.

In some embodiments, the formulation for subcutaneous injection comprising R-ketamine comprises R-ketamine at a concentration between about 70 mg/mL and about 80 mg/mL.

In some embodiments, the formulation for subcutaneous injection comprising R-ketamine comprises R-ketamine at a concentration between about 60 mg/mL and about 70 mg/mL.

In some embodiments, the formulation for subcutaneous injection comprising R-ketamine comprises R-ketamine at a concentration between about 95 mg/mL and about 110 mg/mL.

In some embodiments, the formulation for subcutaneous injection comprising R-ketamine comprises R-ketamine at a concentration between about 90 mg/mL and about 105 mg/mL.

In some embodiments, the formulation for subcutaneous injection comprising R-ketamine comprises R-ketamine at a concentration between about 95 mg/mL and about 105 mg/mL.

In some embodiments, the formulation for subcutaneous injection comprising R-ketamine comprises R-ketamine at a concentration of about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 105 mg/mL or about 110 mg/mL.

In some embodiments, the formulation for subcutaneous injection comprising R-ketamine comprises R-ketamine at a concentration of about 60 mg/mL.

In some embodiments, the formulation for subcutaneous injection comprising R-ketamine comprises R-ketamine at a concentration of about 65 mg/mL.

In some embodiments, the formulation for subcutaneous injection comprising R-ketamine comprises R-ketamine at a concentration of about 70 mg/mL.

In some embodiments, the formulation for subcutaneous injection comprising R-ketamine comprises R-ketamine at a concentration of about 75 mg/mL.

In some embodiments, the formulation for subcutaneous injection comprising R-ketamine comprises R-ketamine at a concentration of about 80 mg/mL.

In some embodiments, the formulation for subcutaneous injection comprising R-ketamine comprises R-ketamine at a concentration of about 85 mg/mL.

In some embodiments, the formulation for subcutaneous injection comprising R-ketamine comprises R-ketamine at a concentration of about 90 mg/mL.

In some embodiments, the formulation for subcutaneous injection comprising R-ketamine comprises R-ketamine at a concentration of about 95 mg/mL.

In some embodiments, the formulation for subcutaneous injection comprising R-ketamine comprises R-ketamine at a concentration of about 100 mg/mL.

In some embodiments, the formulation for subcutaneous injection comprising R-ketamine comprises R-ketamine at a concentration of about 105 mg/mL.

In some embodiments, the formulation for subcutaneous injection comprising R-ketamine comprises R-ketamine at a concentration of about 110 mg/mL.

In some embodiments, the formulation comprises substantially pure R-ketamine. In some embodiments, the formulation is substantially free of S-ketamine. In some embodiments, over 90%, over 95%, over 96%, over 97%, over 98%, or over 99% of the ketamine is R-ketamine. Alternatively, or in addition, R-ketamine that is substantially free of S-ketamine refers to R-ketamine in which S-ketamine is below the limit of detection using conventional methods in the art. As a further alternative R-ketamine that is substantially free of S-ketamine refers to R-ketamine in which the amount of S-ketamine is such that the side effects associated with S(+)-ketamine are substantially reduced, or not present when the composition is administered to a subject. Exemplary side effects include, but are not limited to psychotomimetic effects, such as alteration of perception, mood, thought or mental state, for example anhedonia or negative affect. Additional side effects include, somnolence, dizziness, headache, dysarthria, paraesthesia, balance disorder, hypoaesthesia, lethargy, memory impairment, sedation, sensory disturbance, slow speech, derealization, confusional state, aversion, bruxism, dissociation, euphoria, logorrhoea and altered time perception.

In some embodiments, over 90% of the ketamine is R-ketamine. In some embodiments, over 95% of the ketamine is R-ketamine. In some embodiments, over 96% of the ketamine is R-ketamine. In some embodiments, over 97% of the ketamine is R-ketamine. In some embodiments, over 98% of the ketamine is R-ketamine. In some embodiments, over 99% of the ketamine is R-ketamine. In some embodiments, over 99.5% of the ketamine is R-ketamine. In some embodiments, over 99.9% of the ketamine is R-ketamine.

In some embodiments, the formulation is a liquid formulation for subcutaneous administration comprising R-ketamine. In some embodiments, the formulation is a liquid formulation for subcutaneous administration comprising non-ionized R-ketamine (i.e., free base). In some embodiments, the formulation is a liquid formulation for subcutaneous administration comprising R-ketamine in a mixture of ionized and non-ionized (free base) forms. In some embodiments, the formulation is a liquid formulation for subcutaneous administration comprising R-ketamine in an ionized form. In some embodiments, the formulation is a liquid formulation for subcutaneous administration comprising R-ketamine in a salt form.

In some embodiments, the pH of the injectable formulation of the present disclosure may be adjusted to a suitable pH. In some embodiments, the formulation pH is adjusted using a weak base. In some embodiments, the formulation pH is adjusted using a dilute base. In some embodiments, the formulation pH is adjusted using a strong base. In some embodiments, the formulation pH is adjusted using a weak and dilute base. In some embodiments, the formulation pH is adjusted using a strong and dilute base. In some embodiments, the formulation pH is adjusted using NaOH. In some embodiments, the formulation pH is adjusted using KOH. In some embodiments, the formulation pH is adjusted using 1N NaOH. In some embodiments, the formulation pH is adjusted using 1N KOH. In some embodiments, the formulation pH is adjusted using 0.5N NaOH. In some embodiments, the formulation pH is adjusted using 0.5N KOH.

In some embodiments, the formulation pH is adjusted using a weak acid. In some embodiments, the formulation pH is adjusted using a dilute acid. In some embodiments, the formulation pH is adjusted using a strong acid. In some embodiments, the formulation pH is adjusted using a weak and dilute acid. In some embodiments, the formulation pH is adjusted using a strong and dilute acid.

In some embodiments, the method further comprises adjusting the pH of the pharmaceutical composition. In some embodiments, only a minimal adjustment of the pH is necessary. In some embodiments, the pH is adjusted with a strong base. In some embodiments, the pH is adjusted with sodium hydroxide, potassium hydroxide, barium hydroxide, cesium hydroxide, strontium hydroxide magnesium hydroxide, calcium hydroxide, lithium hydroxide, or rubidium hydroxide.

In some embodiments, the pH is adjusted with sodium hydroxide. In some embodiments, the pH is adjusted to a desired pH. In some embodiments, the formulation is adjusted to a pH of about 5.0. In some embodiments, the formulation is adjusted to a pH of about 5.1. In some embodiments, the formulation is adjusted to a pH of about 5.2. In some embodiments, the formulation is adjusted to a pH of about 5.3. In some embodiments, the formulation is adjusted to a pH of about 5.4. In some embodiments, the formulation is adjusted to a pH of about 5.5. In some embodiments, the formulation is adjusted to a pH of about 5.6. In some embodiments, the formulation is adjusted to a pH of about 5.70. In some embodiments, the formulation is adjusted to a pH of about 5.71. In some embodiments, the formulation is adjusted to a pH of about 5.72. In some embodiments, the formulation is adjusted to a pH of about 5.73. In some embodiments, the formulation is adjusted to a pH of about 5.74. In some embodiments, the formulation is adjusted to a pH of about 5.75. In some embodiments, the formulation is adjusted to a pH of about 5.76. In some embodiments, the formulation is adjusted to a pH of about 5.77. In some embodiments, the formulation is adjusted to a pH of about 5.78. In some embodiments, the formulation is adjusted to a pH of about 5.79. In some embodiments, the formulation is adjusted to a pH of about 5.8. In some embodiments, the formulation is adjusted to a pH of about 5.9. In some embodiments, the formulation is adjusted to a pH of about 6.0. In some embodiments, the formulation is adjusted to a pH of between 5.0 and 6.0.

In some embodiments, the pH of the formulation is between about 5.0 and about 6.0. In some embodiments, the pH of the formulation is between about 5.1 and about 6.0. In some embodiments, the pH of the formulation is between about 5.2 and about 6.0. In some embodiments, the pH of the formulation is between about 5.3 and about 6.0. In some embodiments, the pH of the formulation is between about 5.4 and about 6.0. In some embodiments, the pH of the formulation is between about 5.5 and about 6.0. In some embodiments, the pH of the formulation is between about 5.6 and about 6.0. In some embodiments, the pH of the formulation is between about 5.7 and about 6.0. In some embodiments, the pH of the formulation is between about 5.8 and about 6.0. In some embodiments, the pH of the formulation is between about 5.9 and about 6.0.

In some embodiments, the pH of the formulation is between about 5.0 and about 5.9. In some embodiments, the pH of the formulation is between about 5.1 and about 5.9. In some embodiments, the pH of the formulation is between about 5.2 and about 5.9. In some embodiments, the pH of the formulation is between about 5.3 and about 5.9. In some embodiments, the pH of the formulation is between about 5.4 and about 5.9. In some embodiments, the pH of the formulation is between about 5.5 and about 5.9. In some embodiments, the pH of the formulation is between about 5.6 and about 5.9. In some embodiments, the pH of the formulation is between about 5.7 and about 5.9. In some embodiments, the pH of the formulation is between about 5.8 and about 5.9.

In some embodiments, the pH of the formulation is between about 5.0 and about 5.8. In some embodiments, the pH of the formulation is between about 5.1 and about 5.8. In some embodiments, the pH of the formulation is between about 5.2 and about 5.8. In some embodiments, the pH of the formulation is between about 5.3 and about 5.8. In some embodiments, the pH of the formulation is between about 5.4 and about 5.8. In some embodiments, the pH of the formulation is between about 5.5 and about 5.8. In some embodiments, the pH of the formulation is between about 5.6 and about 5.8. In some embodiments, the pH of the formulation is between about 5.7 and about 5.8.

In some embodiments, the pH of the formulation is between about 5.0 and about 5.7. In some embodiments, the pH of the formulation is between about 5.1 and about 5.7. In some embodiments, the pH of the formulation is between about 5.2 and about 5.7. In some embodiments, the pH of the formulation is between about 5.3 and about 5.7. In some embodiments, the pH of the formulation is between about 5.4 and about 5.7. In some embodiments, the pH of the formulation is between about 5.5 and about 5.7. In some embodiments, the pH of the formulation is between about 5.6 and about 5.7.

In some embodiments, the pH of the formulation is between about 5.0 and about 5.6. In some embodiments, the pH of the formulation is between about 5.1 and about 5.6. In some embodiments, the pH of the formulation is between about 5.2 and about 5.6. In some embodiments, the pH of the formulation is between about 5.3 and about 5.6. In some embodiments, the pH of the formulation is between about 5.4 and about 5.6. In some embodiments, the pH of the formulation is between about 5.5 and about 5.6.

In some embodiments, the pH of the formulation is between about 5.0 and about 5.5. In some embodiments, the pH of the formulation is between about 5.1 and about 5.5. In some embodiments, the pH of the formulation is between about 5.2 and about 5.5. In some embodiments, the pH of the formulation is between about 5.3 and about 5.5. In some embodiments, the pH of the formulation is between about 5.4 and about 5.5.

In some embodiments, the pH of the formulation is between about 5.0 and about 5.4. In some embodiments, the pH of the formulation is between about 5.1 and about 5.4. In some embodiments, the pH of the formulation is between about 5.2 and about 5.4. In some embodiments, the pH of the formulation is between about 5.3 and about 5.4.

In some embodiments, the pH of the formulation is between about 5.0 and about 5.3. In some embodiments, the pH of the formulation is between about 5.1 and about 5.3. In some embodiments, the pH of the formulation is between about 5.2 and about 5.3.

In some embodiments, the pH of the formulation is between about 5.0 and about 5.2. In some embodiments, the pH of the formulation is between about 5.1 and about 5.2.

In some embodiments, the pH of the formulation is between about 5.0 and about 5.1.

In some embodiments, the pH of the formulation is about 5.0. In some embodiments, the pH of the formulation is about 5.1. In some embodiments, the pH of the formulation is about 5.2. In some embodiments, the pH of the formulation is about 5.3. In some embodiments, the pH of the formulation is about 5.4. In some embodiments, the pH of the formulation is about 5.5. In some embodiments, the pH of the formulation is about 5.6. In some embodiments, the pH of the formulation is about 5.70. In some embodiments, the pH of the formulation is about 5.71. In some embodiments, the pH of the formulation is about 5.72. In some embodiments, the pH of the formulation is about 5.73. In some embodiments, the pH of the formulation is about 5.74. In some embodiments, the pH of the formulation is about 5.75. In some embodiments, the pH of the formulation is about 5.76. In some embodiments, the pH of the formulation is about 5.77. In some embodiments, the pH of the formulation is about 5.78. In some embodiments, the pH of the formulation is about 5.79. In some embodiments, the pH of the formulation is about 5.8. In some embodiments, the pH of the formulation is about 5.9. In some embodiments, the pH of the formulation is about 6.0.

In some embodiments, the R-ketamine formulation does not comprise a cyclodextrin. In some embodiments, the R-ketamine formulation does not comprise a cyclodextrin or a cyclodextrin derivative.

The injectable composition may be supplied in various delivery forms, e.g. ampoules, pre-filled syringes, needle or needle free auto-injectors, as a small volume infusion or in multi-dose containers with an added preservative.

In some embodiments of the present disclosure, the parenteral composition is formulated as a subcutaneous injection.

Each of the injection formulations of the disclosure (e.g. a subcutaneous injection formulation) also include water, which may be present as a saline solution, to further dilute the formulation to an appropriate volume suitable for injection.

A suitable volume of a R-ketamine formulation of the disclosure for use in an injection device (e.g. for subcutaneous administration) is in the range of about 0.1 mL to about 10 mL. Suitable volumes may include, for example, 0.1 mL, 0.2 mL, 0.3 mL, 0.4 mL, 0.5 mL, 0.6 mL, 0.7 mL, 0.8 mL, 0.9 mL, 1.0 mL, 2.0, mL, 3.0 mL, 4.0 mL, 5.0 mL, 6.0 mL, 7.0 mL, 8.0 mL, 9.0 mL and 10.0 mL and all amounts therebetween.

In some embodiments, the formulation has an osmolality of from about 150 mOsm/kg to about 850 mOsm/kg. In some embodiments, the formulation has an osmolality of from about 175 mOsm/kg to about 850 mOsm/kg. In some embodiments, the formulation has an osmolality of from about 200 mOsm/kg to about 850 mOsm/kg. In some embodiments, the formulation has an osmolality of from about 225 mOsm/kg to about 850 mOsm/kg. In some embodiments, the formulation has an osmolality of from about 250 mOsm/kg to about 850 mOsm/kg. In some embodiments, the formulation has an osmolality of from about 275 mOsm/kg to about 850 mOsm/kg. In some embodiments, the formulation has an osmolality of from about 300 mOsm/kg to about 850 mOsm/kg. In some embodiments, the formulation has an osmolality of from about 325 mOsm/kg to about 850 mOsm/kg. In some embodiments, the formulation has an osmolality of from about 350 mOsm/kg to about 850 mOsm/kg. In some embodiments, the formulation has an osmolality of from about 375 mOsm/kg to about 850 mOsm/kg. In some embodiments, the formulation has an osmolality of from about 400 mOsm/kg to about 850 mOsm/kg. In some embodiments, the formulation has an osmolality of from about 300 mOsm/kg to about 450 mOsm/kg. In some embodiments, the formulation has an osmolality of from about 475 mOsm/kg to about 850 mOsm/kg. In some embodiments, the formulation has an osmolality of from about 500 mOsm/kg to about 850 mOsm/kg.

In some embodiments, the formulation has an osmolality of at least about 150 mOsm/kg. In some embodiments, the formulation has an osmolality of at least about 175 mOsm/kg. In some embodiments, the formulation has an osmolality of at least about 200 mOsm/kg. In some embodiments, the formulation has an osmolality of at least about 225 mOsm/kg. In some embodiments, the formulation has an osmolality of at least about 250 mOsm/kg. In some embodiments, the formulation has an osmolality of at least about 275 mOsm/kg. In some embodiments, the formulation has an osmolality of at least about 300 mOsm/kg. In some embodiments, the formulation has an osmolality of at least about 325 mOsm/kg. In some embodiments, the formulation has an osmolality of at least about 350 mOsm/kg. In some embodiments, the formulation has an osmolality of at least about 375 mOsm/kg. In some embodiments, the formulation has an osmolality of at least about 400 mOsm/kg. In some embodiments, the formulation has an osmolality of at least about 425 mOsm/kg. In some embodiments, the formulation has an osmolality of at least about 450 mOsm/kg. In some embodiments, the formulation has an osmolality of at least about 475 mOsm/kg. In some embodiments, the formulation has an osmolality of at least about 500 mOsm/kg.

In some embodiments, the formulation has an osmolality of about <850 mOsm/kg. In some embodiments, the formulation has an osmolality of about <825 mOsm/kg. In some embodiments, the formulation has an osmolality of about <800 mOsm/kg. In some embodiments, the formulation has an osmolality of about <775 mOsm/kg. In some embodiments, the formulation has an osmolality of about <750 mOsm/kg. In some embodiments, the formulation has an osmolality of about <725 mOsm/kg. In some embodiments, the formulation has an osmolality of about <700 mOsm/kg. In some embodiments, the formulation has an osmolality of about <675 mOsm/kg. In some embodiments, the formulation has an osmolality of about <650 mOsm/kg. In some embodiments, the formulation has an osmolality of about <625 mOsm/kg. In some embodiments, the formulation has an osmolality of about <600 mOsm/kg. In some embodiments, the formulation has an osmolality of about <575 mOsm/kg. In some embodiments, the formulation has an osmolality of about <550 mOsm/kg. In some embodiments, the formulation has an osmolality of about <525 mOsm/kg. In some embodiments, the formulation has an osmolality of about <500 mOsm/kg. In some embodiments, the formulation has an osmolality of about <450 mOsm/kg. In some embodiments, the formulation has an osmolality of about <400 mOsm/kg. In some embodiments, the formulation has an osmolality of about <350 mOsm/kg. In some embodiments, the formulation has an osmolality of about <300 mOsm/kg. In some embodiments, the formulation has an osmolality of about <250 mOsm/kg.

In some embodiments, the formulation has an osmolality of about 300 mOsm/kg to about 850 mOsm/kg. In some embodiments, the formulation has an osmolality of about 300 mOsm/kg to about 350 mOsm/kg, about 300 mOsm/kg to about 400 mOsm/kg, about 300 mOsm/kg to about 450 mOsm/kg, about 300 mOsm/kg to about 500 mOsm/kg, about 300 mOsm/kg to about 550 mOsm/kg, about 300 mOsm/kg to about 600 mOsm/kg, about 300 mOsm/kg to about 650 mOsm/kg, about 300 mOsm/kg to about 700 mOsm/kg, about 300 mOsm/kg to about 750 mOsm/kg, about 300 mOsm/kg to about 800 mOsm/kg, about 300 mOsm/kg to about 850 mOsm/kg, about 350 mOsm/kg to about 400 mOsm/kg, about 350 mOsm/kg to about 450 mOsm/kg, about 350 mOsm/kg to about 500 mOsm/kg, about 350 mOsm/kg to about 550 mOsm/kg, about 350 mOsm/kg to about 600 mOsm/kg, about 350 mOsm/kg to about 650 mOsm/kg, about 350 mOsm/kg to about 700 mOsm/kg, about 350 mOsm/kg to about 750 mOsm/kg, about 350 mOsm/kg to about 800 mOsm/kg, about 350 mOsm/kg to about 850 mOsm/kg, about 400 mOsm/kg to about 450 mOsm/kg, about 400 mOsm/kg to about 500 mOsm/kg, about 400 mOsm/kg to about 550 mOsm/kg, about 400 mOsm/kg to about 600 mOsm/kg, about 400 mOsm/kg to about 650 mOsm/kg, about 400 mOsm/kg to about 700 mOsm/kg, about 400 mOsm/kg to about 750 mOsm/kg, about 400 mOsm/kg to about 800 mOsm/kg, about 400 mOsm/kg to about 850 mOsm/kg, about 450 mOsm/kg to about 500 mOsm/kg, about 450 mOsm/kg to about 550 mOsm/kg, about 450 mOsm/kg to about 600 mOsm/kg, about 450 mOsm/kg to about 650 mOsm/kg, about 450 mOsm/kg to about 700 mOsm/kg, about 450 mOsm/kg to about 750 mOsm/kg, about 450 mOsm/kg to about 800 mOsm/kg, about 450 mOsm/kg to about 850 mOsm/kg, about 500 mOsm/kg to about 550 mOsm/kg, about 500 mOsm/kg to about 600 mOsm/kg, about 500 mOsm/kg to about 650 mOsm/kg, about 500 mOsm/kg to about 700 mOsm/kg, about 500 mOsm/kg to about 750 mOsm/kg, about 500 mOsm/kg to about 800 mOsm/kg, about 500 mOsm/kg to about 850 mOsm/kg, about 550 mOsm/kg to about 600 mOsm/kg, about 550 mOsm/kg to about 650 mOsm/kg, about 550 mOsm/kg to about 700 mOsm/kg, about 550 mOsm/kg to about 750 mOsm/kg, about 550 mOsm/kg to about 800 mOsm/kg, about 550 mOsm/kg to about 850 mOsm/kg, about 600 mOsm/kg to about 650 mOsm/kg, about 600 mOsm/kg to about 700 mOsm/kg, about 600 mOsm/kg to about 750 mOsm/kg, about 600 mOsm/kg to about 800 mOsm/kg, about 600 mOsm/kg to about 850 mOsm/kg, about 650 mOsm/kg to about 700 mOsm/kg, about 650 mOsm/kg to about 750 mOsm/kg, about 650 mOsm/kg to about 800 mOsm/kg, about 650 mOsm/kg to about 850 mOsm/kg, about 700 mOsm/kg to about 750 mOsm/kg, about 700 mOsm/kg to about 800 mOsm/kg, about 700 mOsm/kg to about 850 mOsm/kg, about 750 mOsm/kg to about 800 mOsm/kg, about 750 mOsm/kg to about 850 mOsm/kg, or about 800 mOsm/kg to about 850 mOsm/kg. In some embodiments, the formulation has an osmolality of about 300 mOsm/kg, about 350 mOsm/kg, about 400 mOsm/kg, about 450 mOsm/kg, about 500 mOsm/kg, about 550 mOsm/kg, about 600 mOsm/kg, about 650 mOsm/kg, about 700 mOsm/kg, about 750 mOsm/kg, about 800 mOsm/kg, or about 850 mOsm/kg. In some embodiments, the formulation has an osmolality of at least about 300 mOsm/kg, about 350 mOsm/kg, about 400 mOsm/kg, about 450 mOsm/kg, about 500 mOsm/kg, about 550 mOsm/kg, about 600 mOsm/kg, about 650 mOsm/kg, about 700 mOsm/kg, about 750 mOsm/kg, or about 800 mOsm/kg. In some embodiments, the formulation has an osmolality of at most about 350 mOsm/kg, about 400 mOsm/kg, about 450 mOsm/kg, about 500 mOsm/kg, about 550 mOsm/kg, about 600 mOsm/kg, about 650 mOsm/kg, about 700 mOsm/kg, about 750 mOsm/kg, about 800 mOsm/kg, or about 850 mOsm/kg.

In some embodiments, the formulation is isotonic.

In some embodiments, the formulation has an osmolality of about 500 mOsm/kg.

### Stability

In some embodiments, the formulation remains stable for more than about four weeks, about five weeks, about six weeks, about seven weeks, about eight weeks, about nine weeks, about ten weeks, about eleven weeks, about twelve weeks, about thirteen weeks, about fourteen weeks, or about fifteen weeks.

In some embodiments, the formulation remains stable for more than about 1 month, 2 months, 3 months, 4 months, 5 months, 6 months, 7 months, 8 months, 9 months, 10 months, 11 months, about 12 months, 13 months, 14 months, 15 months, 16 months, 17 months, 18 months, 19 months, 20 months, 21 months, 22 months, 23 months, or 24 months.

In some embodiments, the concentration of R-ketamine in the formulation is stable for more than about four weeks, about five weeks, about six weeks, about seven weeks, about eight weeks, about nine weeks, about ten weeks, about eleven weeks, about twelve weeks, about thirteen weeks, about fourteen weeks, or about fifteen weeks.

In some embodiments, the concentration of R-ketamine in the formulation is stable for more than about 1 month, 2 months, 3 months, 4 months, 5 months, 6 months, 7 months, 8 months, 9 months, 10 months, 11 months, about 12 months, 13 months, 14 months, 15 months, 16 months, 17 months, 18 months, 19 months, 20 months, 21 months, 22 months, 23 months, or 24 months.

In some embodiments, the impurities in the formulation are stable for more than about four weeks, about five weeks, about six weeks, about seven weeks, about eight weeks, about nine weeks, about ten weeks, about eleven weeks, about twelve weeks, about thirteen weeks, about fourteen weeks, or about fifteen weeks.

In some embodiments, the impurities in the formulation are stable for more than about 1 month, 2 months, 3 months, 4 months, 5 months, 6 months, 7 months, 8 months, 9 months, 10 months, 11 months, about 12 months, 13 months, 14 months, 15 months, 16 months, 17 months, 18 months, 19 months, 20 months, 21 months, 22 months, 23 months, or 24 months.

In some embodiments, the osmolality in the formulation is stable for more than about four weeks, about five weeks, about six weeks, about seven weeks, about eight weeks, about nine weeks, about ten weeks, about eleven weeks, about twelve weeks, about thirteen weeks, about fourteen weeks, or about fifteen weeks.

In some embodiments, the osmolality in the formulation is stable for more than about 1 month, 2 months, 3 months, 4 months, 5 months, 6 months, 7 months, 8 months, 9 months, 10 months, 11 months, about 12 months, 13 months, 14 months, 15 months, 16 months, 17 months, 18 months, 19 months, 20 months, 21 months, 22 months, 23 months, or 24 months.

In some embodiments, the pH of the formulation is stable for more than about four weeks, about five weeks, about six weeks, about seven weeks, about eight weeks, about nine weeks, about ten weeks, about eleven weeks, about twelve weeks, about thirteen weeks, about fourteen weeks, or about fifteen weeks.

In some embodiments, the pH of the formulation is stable for more than about 1 month, 2 months, 3 months, 4 months, 5 months, 6 months, 7 months, 8 months, 9 months, 10 months, 11 months, about 12 months, 13 months, 14 months, 15 months, 16 months, 17 months, 18 months, 19 months, 20 months, 21 months, 22 months, 23 months, or 24 months.

In some embodiments, the formulation is stable in the presence of light. In some embodiments, the formulation is stable in the presence of heat. In some embodiments, the formulation is stable at room temperature. In some embodiments, the formulation is stable in the presence of heat.

In some embodiments, the term stable means the percentage, volume, or concentration of impurities, pH, R-ketamine, osmolality, etc. does not change more than about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or about 10% from the original percentage, volume, or concentration.

### Carrier Fluid

In some embodiments, the R-ketamine subcutaneous formulation comprises a carrier liquid. In some embodiments, the R-ketamine subcutaneous formulation comprises a carrier liquid, wherein the carrier fluid is water. In some embodiments, the R-ketamine subcutaneous formulation comprises a carrier liquid, wherein the carrier fluid is water for injection (WFI). In some embodiments, the R-ketamine subcutaneous formulation comprises a carrier liquid, wherein the carrier fluid is a buffering agent.

In some embodiments, the R-ketamine subcutaneous formulation comprises water for injection. In some embodiments, the R-ketamine subcutaneous formulation comprises water for injection and a buffer. In some embodiments, the R-ketamine subcutaneous formulation comprises a buffer.

In some embodiments, the R-ketamine subcutaneous formulation comprises a succinate buffer.

In some embodiments, the R-ketamine subcutaneous formulation comprises a tartrate buffer.

In some embodiments, the R-ketamine subcutaneous formulation comprises a maleate buffer.

In some embodiments, the R-ketamine subcutaneous formulation comprises a fumarate buffer.

In some embodiments, the R-ketamine subcutaneous formulation comprises a citrate buffer.

In some embodiments, the R-ketamine subcutaneous formulation comprises an acetate buffer.

In some embodiments, the R-ketamine subcutaneous formulation comprises water for injection and a maleate buffer.

In some embodiments, the R-ketamine subcutaneous formulation comprises water for injection and a fumarate buffer.

In some embodiments, the R-ketamine subcutaneous formulation comprises water for injection and a citrate buffer.

In some embodiments, the R-ketamine subcutaneous formulation comprises water for injection and an acetate buffer.

### Processes for Preparing the Formulation

In some embodiments, R-ketamine, or pharmaceutically acceptable salt thereof, is added to water for injection and the pH is adjusted slowly using a dilute base such that the final concentration of R-ketamine is between about 60 mg/mL and about 110 mg/mL. In some embodiments, the R-ketamine used to prepare the formulation is selected from any of the salts or salt forms described in International Application No. PCT/US2022/046412. In some embodiments, a particular amount of the R-ketamine, R-ketamine salt, or R-ketamine salt form is added to obtain the free base equivalents as disclosed herein.

In some embodiments, the pH of the R-ketamine formulation is increased using an inorganic base. In some embodiments, the pH of the R-ketamine formulation is increased using an inorganic base at a concentration of 1N. In some embodiments, the pH of the R-ketamine formulation is increased using an inorganic base at a concentration of 0.5N. In some embodiments, the pH of the R-ketamine formulation is increased using an inorganic base at a concentration of 0.15N. In some embodiments, the pH of the R-ketamine formulation is titrated.

In some embodiments, the process discussed herein for preparing a formulation for subcutaneous administration comprising R-ketamine facilitates increased solubility of the R-ketamine. In some embodiments, the pH is adjusted slowly. In some embodiments, the pH is adjusted slowly with a dilute acid or a dilute base. In some embodiments, the pH is adjusted in a way that minimizes localized fluctuations of pH. In some embodiments, the pH is adjusted such that micro-changes in pH in the solution is minimized. In some embodiments, the pH is adjusted slowly using a dilute acid or a dilute base such that micro-changes in pH in the solution is minimized. In some embodiments, the pH is adjusted slowly using a dilute acid or a dilute base such that micro-changes in pH in the solution is minimized which improves solubility of the R-ketamine in the formulation.

The injectable compositions of the present disclosure can be manufactured by the skilled person by use of standard methods and conventional techniques appropriate to the desired formulation. The formulation for intramuscular or subcutaneous administration of the present disclosure can be packed and/or stored in a suitable container, including, without limitation, syringes, ampoules, vials, including sealed vials such as vials the openings of which are sealed with syringe pierceable septa or sure-seals caps, and the like. In some embodiments, the formulation is pre-filled in disposable syringes for self-administration by patients, with or without an auto-injector. Each container can contain R-ketamine or a pharmaceutically acceptable salt thereof in desired dosage amounts.

To further minimize oxidative degradation of the active ingredients, the container may be filled with an inert gas, such as nitrogen and/or carbon dioxide, which is otherwise oxygen-free. It is further contemplated that the container(s) may be enclosed within a sealed package, from which oxygen has been excluded. This may be accomplished by vacuum packaging or by displacing oxygen with a blanket or purge of nitrogen, carbon dioxide, or other oxygen-free inert gas. After sealing the seal, the packaging materials themselves should be relatively impermeable to the diffusion of oxygen. Also, the packages should be opaque to ordinary light, as light may induce the decomposition of R-ketamine. Standard methods for sealing and packaging the various containers described herein are well known in the art and can be used in conjunction with packaging and/or storing the dosage forms of the present disclosure.

In some embodiments, the R-ketamine formulation is contained in a “unit dosage form.” The phrase “unit dosage form” refers to physically discrete units, each unit including a predetermined amount of R-ketamine, sufficient to produce the desired effect. It will be appreciated that the parameters of a unit dosage form will depend on the concentration of R-ketamine in the formulation, and the effect to be achieved.

### Methods of Use

The R-ketamine formulations of the present disclosure are useful in the prevention and/or treatment of various diseases or conditions.

The R-ketamine formulations described herein are useful as a neuroprotective, preventive, or therapeutic agent for diseases or conditions associated with glutamatergic transmission, in particular, glutamatergic neurotransmission via an N-methyl-D-aspartate (hereinafter abbreviated as NMDA) receptor. Growing evidence suggests that abnormal glutamatergic transmission, e.g., by the NMDA receptor, is associated with the pathophysiology of mood disorders, and that the NMDA receptor also plays key roles in neurobiology.

The R-ketamine formulations described herein are useful as a neuroprotective, preventive, or therapeutic agent for cognitive dysfunctions such as mood disorders, bipolar disorder, major depressive disorder, general anxiety disorder, panic disorder, obsessive compulsive disorder (OCD), post-traumatic stress disorder (PTSD), attention deficit hyperactivity disorder (ADHD), autism spectrum disorder (ASD), eating disorders, and substance use disorders (drug dependency).

The R-ketamine formulations described herein are also useful treating neurodegenerative diseases and a number of medical diseases, including, but not limited to, cardiovascular diseases, cancer (also referred to as malignant tumors), inflammatory diseases, bone diseases and the like). Exemplary inflammatory and bone diseases include ulcerative colitis, Crohn's disease, rheumatoid arthritis, ankylosing spondylitis, insulin-dependent diabetes, Addison's disease, Goodpasture syndrome, IgA nephropathy, interstitial nephritis, Sjögren's syndrome, autoimmune pancreatitis, psoriasis, atopic dermatitis, pneumonia, chronic bronchitis, bronchial asthma, systemic lupus erythematosus (SLE), scleroderma, or delirium, and the bone disease is osteoporosis, osteolytic bone metastasis, and Paget's disease of bone. Since inflammatory diseases are chronic diseases that progress over a long period of time (years), beginning treatment early may prevent the progression of symptoms. Furthermore, by administering the R-ketamine formulations described herein to patients having a genetic background that is potentially susceptible to inflammatory diseases before they exhibit symptoms, the R-ketamine formulations described herein may also be used to prevent the onset of the inflammatory or bone disease.

The R-ketamine formulations described herein are also useful in treating neurodegenerative diseases, such as Parkinson's disease, Alzheimer's disease, Lewy body dementia and the like. In some embodiments, the R-ketamine formulations described herein treat one or more symptoms of the neurodegenerative disease.

The R-ketamine formulations described herein are also useful in treating neurodevelopmental conditions or disorders, for example childhood or fetal neurodevelopmental disorders. Exemplary neurodevelopmental disorders include schizophrenia, autism spectrum disorder, attention-deficit/hyperactivity disorder, autism spectrum disorder and other learning disorders. Symptoms include cognitive impairment, such as impaired attention, reduced linguistic fluidity, reduced learning and retention of linguistic information, reduced processing speed, reduced declarative memory, impaired working memory, a reduced executive function, or a combination thereof. In some embodiments, the R-ketamine formulations described herein treat one or more symptoms of a neurodevelopmental disorder. The R-ketamine formulations described herein are useful as a neuroprotective, preventive, or therapeutic agent for diseases or conditions associated with brain dopamine loss. The R-ketamine formulations described herein may be used for preventing or treating diseases or conditions associated with a decrease of dopamine transporters (DAT). In particular, examples of dysfunctions or conditions of the brain dopamine nervous system include substance use disorders (drug dependency) known in abusers of stimulant drugs or cocaine, and R-ketamine is considered to be effective as a preventive or therapeutic drug for substance use disorders (drug dependency). It has been reported that dopamine transporter (DAT) is decreased in the brains of cocaine abusers and stimulant drug (methamphetamine) users, and it has been pointed out that the decrease in DAT is associated with cognitive dysfunction. In addition, the decrease in DAT in substance use disorder patients is similar to the decrease in DAT seen in some neurodegenerative diseases, for example Parkinson's disease patients.

In addition, the R-ketamine formulations described herein may be involved in the signal mediated by the brain-derived neurotrophic factor BDNF which leads to it having a neuroprotective effect. Due to these neuroprotective effects, the R-ketamine formulations described herein may be used not only for the prevention or treatment of depressive symptoms and neurodegenerative diseases, but also for the prevention or treatment of cognitive dysfunctions described above as the description of the term “cognitive dysfunction.”

Accordingly, the disclosure provides compositions comprising the R-ketamine formulations described herein for use in the prevention or treatment of a neurodevelopmental disorder, neurodegenerative disease, inflammatory or bone disease, or a cognitive dysfunction.

The disclosure provides compositions comprising the R-ketamine formulations described herein for use in the manufacture of a medicament for the prevention or treatment of a neurodevelopmental disorder, neurodegenerative disease, inflammatory or bone disease, or a cognitive dysfunction.

The disclosure provides methods of preventing or treating a neurodevelopmental disorder, neurodegenerative disease, inflammatory or bone disease, or a cognitive dysfunction, comprising administering a therapeutically effective amount of a composition comprising an R-ketamine formulation described herein.

By using the R-ketamine formulations described herein as a preventive or therapeutic agent for a neurodevelopmental disorder, neurodegenerative disease, inflammatory or bone disease, or a cognitive dysfunction, it is possible to prevent the onset of the disease or disorder and to exert therapeutic effects such as alleviating and improving symptoms in patients suffering from a neurodevelopmental disorder, neurodegenerative disease, inflammatory or bone disease, or cognitive dysfunction. Since many of the disorders, diseases and dysfunctions described herein progress over a long period (measured in years), early treatment may also prevent the progression of, or reduce the severity of symptoms.

In addition, it may also be used for a patient with a genetic background potentially predisposing him or her to a neurodevelopmental disorder, neurodegenerative disease, inflammatory or bone disease, or a cognitive dysfunction to prevent the onset of the disorder, disease or dysfunction by administering it before the symptoms are exhibited.

In the present disclosure, the R-ketamine formulations described herein is used for the prevention or treatment of a neurodevelopmental disorder, neurodegenerative disease, inflammatory or bone disease, or a cognitive dysfunction, the administration of the drug is scheduled to be a long period of time.

Therefore, by using the R-ketamine formulations described herein, it is possible to administer the drug for a long period of time without exerting side effects, so the R-ketamine formulations described herein may be used as a preventive or therapeutic agent for a neurodevelopmental disorder, neurodegenerative disease, inflammatory or bone disease, or a cognitive dysfunction.

The R-ketamine formulations described herein are useful as a preventative agent for a neurodevelopmental disorder, neurodegenerative disease, inflammatory or bone disease, or a cognitive dysfunction by preventing the onset of the disorder, disease or dysfunction, or as an agent for preventing the progression of the symptoms of the disorder, disease or dysfunction. In addition, the therapeutic agent may have a therapeutic effect of preventing the progression of the symptoms, and alleviating or improving the symptoms.

The processes regarding the R-ketamine formulations described herein are useful the prevention and/or treatment of one or more symptoms associated with any of the disorder, disease or dysfunctions described herein, for example depressive symptoms. In some embodiments, depressive symptoms include but are not limited a lack of interest in activities, changes in sleep pattern, changes in appetite, feelings of guilt, feelings of despair, lack of energy, trouble concentrating, stress, low mood or mood depression, impaired motivation, cognitive impairment, diminished ability to think, anxiety, insomnia, anhedonia and negative affect, anorexia, fatigue and suicidal thoughts. The processes for preparing R-ketamine formulations of the present disclosure are useful in preparing a medicament for the prevention and/or treatment of depression.

The R-ketamine formulations described herein are useful the prevention and/or treatment of a substance use disorder in a subject. In some embodiments, the substance use disorder comprises abuse of alcohol, marijuana, synthetic cannabinoids, opioids, stimulants, barbiturates, benzodiazepines, dextromethorphan (DXM), a sleep medication, khat, synthetic cathinones, cocaine, 3,4-methylenedioxymethamphetamine (MDMA), phencyclidine (PCP), lysergic acid diethylamide (LSD), psilocybin, an inhalant, Rohypnol, gamma-hydroxybutyric acid (GHB), N,N-Dimethyltryptamine (DMT), ayahuasea, mescaline, salvia, or nicotine.

The R-ketamine formulations described herein are useful for the prevention and/or treatment of a substance use withdrawal symptom in a subject. In some embodiments, the substance use withdrawal symptom comprises a symptom of withdrawal from alcohol, marijuana, synthetic cannabinoids, opioids, stimulants, barbiturates, benzodiazepines, dextromethorphan (DXM), a sleep medication, khat, synthetic cathinones, cocaine, 3,4-methylenedioxymethamphetamine (MDMA), phencyclidine (PCP), lysergic acid diethylamide (LSD), psilocybin, an inhalant, Rohypnol, gamma-hydroxybutyric acid (GHB), N,N-Dimethyltryptamine (DMT), ayahuasca, mescaline, salvia, or nicotine.

Additional disorders, and symptoms thereof that can be treated by the R-ketamine formulations described herein are described in WO 2015/037248 (PCT/JP2014/004730), WO 2019/213551 (PCT/US2019/030644), WO 2019/065900 (PCT/JP2018/036079), WO 2019/160057 (PCT/JP2019/005415), and WO 2020/138491 (PCT/JP2019/051605) which are incorporated by reference in their entireties.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

All patents, patent applications, and publications mentioned herein are hereby incorporated by reference in their entireties. However, where a patent, patent application, or publication containing express definitions is incorporated by reference, those express definitions should be understood to apply to the incorporated patent, patent application, or publication in which they are found, and not to the remainder of the text of this application, in particular the claims of this application.

### Kits

The disclosure provides kits comprising the R-ketamine formulations and pharmaceutical compositions described herein.

In some embodiments, the R-ketamine formulation or pharmaceutical composition is stored in a reservoir of a drug delivery device. In some embodiments, the formulation is stored in a cartridge that is insertable and/or attachable to a drug delivery device. In some embodiments, the R-ketamine formulation is stored in a reservoir of a drug delivery device comprising a needle configured for subcutaneous injection. In some embodiments, the cartridge and/or drug delivery device comprises a product label for subcutaneous injection. In some embodiments, disclosed herein is a kit comprising a product label for subcutaneous injection.

### EXAMPLES

R-ketamine is the R-(−) enantiomer of ketamine (IUPAC name: 2-(2-chlorophenyl)-2-(methylamino)cyclohexan-1-one). R-ketamine is being investigated for the treatment of several neurological conditions. Due to the poor solubility of the free base in water, it is usually used as a hydrochloride salt, which exhibits solubilities greater than 140 mg/mL in water at a pH>5. R-ketamine properties as used herein: molecular weight: 274.18 g/mol as a hydrochloride salt; molecular weight as free base: 237.75 g/mol; molecular formula: C₁₃H₁₇Cl₂NO.

TABLE 1
General Properties of R-ketamine

| Property | Description |
|---|---|
| Appearance: | White crystalline powder |
| Optical rotation | −88.0° to −95.0° |
| Stereochemistry | R enantiomer; chiral center at |
| | C-2 of the cyclohexanone ring |
| Melting range: | Melting onset 267° C. with |
| | peak at 270° C. (DSC) |
| Log P: | 2.18 |
| Solubility profile: | Table 2 |
| Hygroscopicity: | Not hygroscopic, no moisture uptake |
| | between 50% RH to 90% RH in |
| | DVS experiments |
| pKa | 7.5 at 25° C. |
| pH | 3.5 to 4.5 in aqueous solution (10% w/v) |
| Physical Form | Crystalline |

TABLE 2
Solubility Profile

| | Approximate | Solubility |
|---|---|---|
| Solvent | Solubility | Classification (EP) |
| Water | >140 mg/mL (pH 4-4.5) | Freely soluble |
| Acetone, acetonitrile, | <1 mg/mL | Very slightly soluble |
| Anisole, ethyl acetate, | | |
| heptane, methyl ethyl | | |
| ketone, methyl iso-butyl | | |
| ketone, tetrahydrofuran, | | |
| methyl tert-butyl ether | | |
| DMSO, Ethanol | ~20 to 30 mg/mL | Sparingly soluble |
| 1-propanol | ~13 to 15 mg/mL | Sparingly soluble |
| Methanol | ~50 to 100 mg/mL | Soluble |
| 2-propanol | ~4 mg/mL | Slightly soluble |

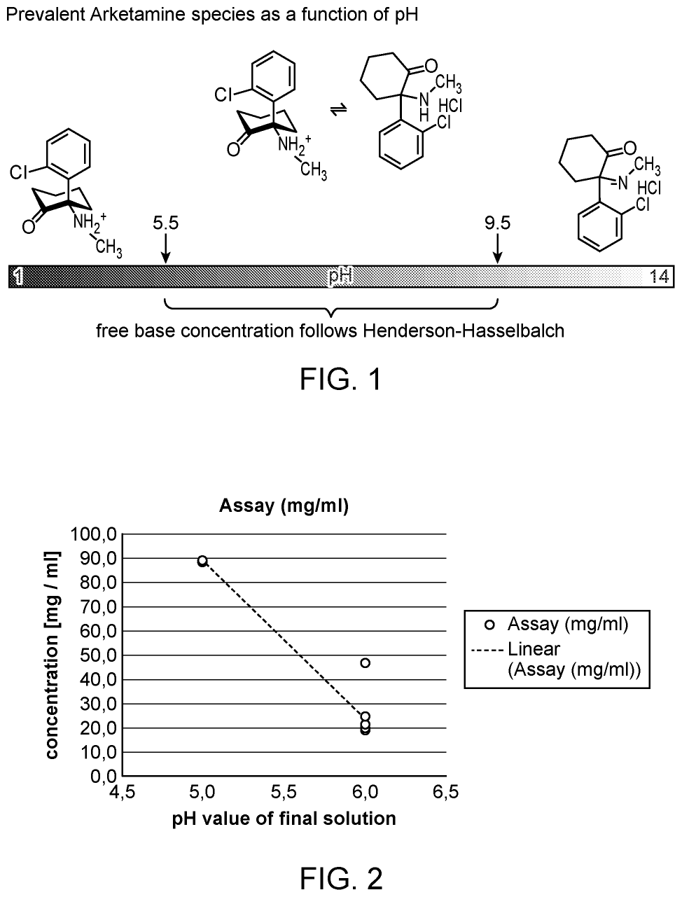

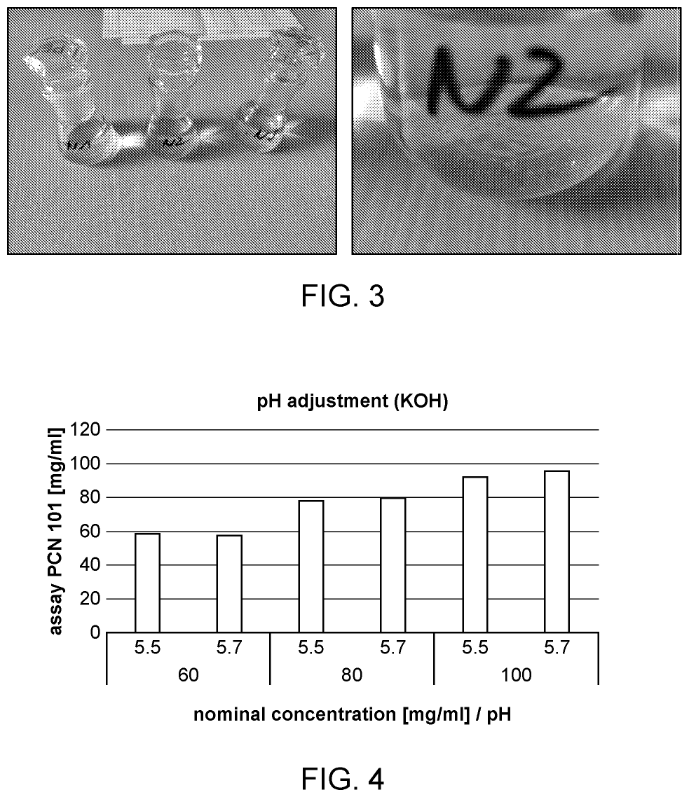

In water, R-ketamine exhibits buffering properties following Henderson-Hasselbalch between pH 5.5 and 9.5, as depicted in FIG. 1. From pH 5.5, the free base consequently precipitates, if this is not prevented by the addition of suitable excipients.

An R-ketamine formulation suitable for subcutaneous injection at a concentration of 60 mg/ml (100 mg/ml) was developed. In addition to the concentration, a pH between 5 and 6 and tonicity as close as possible to physiological values were achieved.

Formation of inclusion complexes between the R-ketamine free base and supplemented cyclodextrins, addition of cosolvents, or buffering of the formulation at suitable pH values were examined. The interaction between the free base present at pH values greater than 5.5 with beta cyclodextrin was investigated using statistical design of experiments. Promising candidates for cosolvent screening were tested for applicability. For the investigation of suitable buffer systems, two compendial buffer systems were employed, and their suitability was tested between pH 5.2 and 5.8.

### Example 1. Cyclodextrin Inclusion

The effect of hydroxypropyl β-cyclodextrin (HPB) on the solubility of R-ketamine at different pH values was investigated. Varied factors were pH of the solution and the concentration of HPB. Response factors were tonicity, which was determined by freezing point depression, and API content, which was measured by HPLC, of a filtered (0.22 μm) solution.

TABLE 3
Factor Settings

| Name | Abbr. | Units | Type | Settings | Transform | Precision |
|---|---|---|---|---|---|---|
| Cyclo- | Cyc | % | Quantitative | 0 to 20 | None | 0.5 |
| dextrin | | | | | | |
| pH | pH | pH | Quantitative | 4.5 to 7.5 | None | 0.075 |

TABLE 4
Tonicity and Assay Requirements

| Name | Abbr. | Units | Min | Target | Max |
|---|---|---|---|---|---|
| Tonicity | Osm | mOsmol | 250 | 300 | 350 |
| Assay | Conc. | mg/ml | 60 | 100 | |

Using the above specifications, the following full factorial design was prepared and tested.

TABLE 5

| Summary of Cyclodextrin/pH Studies | | |
|---|---|---|
| Exp | | |
| No | Cyclodextrin | pH |
| 1 | 0 | 4.5 |
| 2 | 20 | 4.5 |
| 3 | 0 | 7.5 |
| 4 | 20 | 7.5 |
| 5 | 10 | 6 |
| 6 | 10 | 6 |
| 7 | 10 | 6 |

### Experimental Procedure

After dissolving the active ingredient (100 mg/ml, 86.96 mg/ml free base), a defined amount of HPB was added. After complete solution of the two components, the target pH was adjusted with sodium hydroxide solution (1 M). The resulting solution was filtered through a syringe filter (0.22 μm) and analyzed by HPLC and freezing point depression. A separate protocol was prepared for each individual experiment.

### Results

The results (Table 6) confirm the pronounced pH dependence of the solubility of R-ketamine in water, which is apparently not significantly affected by the addition of HPB. The lack of correlation between tonicity and drug content at higher pH values is striking. While this phenomenon was initially attributed to errors in the control strategy, it can now be understood to be due to the volumes of sodium hydroxide used for pH adjustment.

TABLE 6

| Results | | | |
|---|---|---|---|
| Cyclodextrin | pH | Tonicity | Assay |
| 0 | 4.5 | 644 | 88.9 |
| 20 | 4.5 | 638 | 93.9 |
| 0 | 7.5 | 522 | 0.9 |
| 20 | 7.5 | 546 | 11.1 |
| 10 | 6 | 683 | 29.6 |
| 10 | 6 | 680 | 26.8 |
| 10 | 6 | 680 | 26.8 |

### Example 2. Cosolvent Screening

For cosolvent screening, a set of possible candidates was identified. Specifically, 5% sulfobutyl-ether-β-cyclodextrin (SBE7), polyethylene glycol 400 (PEG 400), glycerol, propylene glycol, and 5% polysorbate 80 were to be screened at pH 5 and 6. Unless otherwise specified, cosolvents were screened at 20% and 50% (m/V). As a control, the solubility of R-ketamine in water was studied at pH 5 and 6.

### Experimental Procedure

The test solution consisting of water for injection. The respective cosolvent was prepared and the active substance was dissolved to a nominal concentration of 100 mg/ml (equaling 86.96 mg/ml free base). The pH was then adjusted to pH 5 with 1N NaOH and a sample was obtained under filtration (0.22 μm). Thereafter, the pH was further increased to pH 6 and another sample was taken (also filtered). Both samples were analyzed for assay and tonicity by HPLC and freezing point depression.

### Results

Batch size: 90 g/pH set at end of compounding (1N NaOH/1N HCl)

TABLE 7
Cosolvent Screening

| | | Tonicity | Assay |
|---|---|---|---|
| Description | pH | (mOsmol) | (mg/mL) |
| Water for injection | 5.0 | 651 | 88.6 |
| Water for injection | 6.0 | 568 | 19.7 |
| PEG 20% | 6.0 | 695 | 19.9 |
| PEG 50% | 6.0 | ** | 21.6 |
| Glycerol 20% | 6.0 | 1445 | 19.1 |
| Glycerol 50% | 6.0 | ** | 20.2 |
| Propylene glycol 20% | 6.0 | ** | 24.5 |
| Propylene glycol 50% | 6.0 | ** | 46.5 |
| Tween 5% | 5.0 | 686 | 89.2 |
| Tween 5% | 6.0 | 602 | 18.8 |

** dilution tested, no value obtainable

The use of cosolvents did not lead to the desired effect to reach a solution containing 60 mg/mL of R-ketamine hydrochloride. The interaction between pH optimum below 5 could not be overcome by the different cosolvents in different concentration as above. Only a concentration of 50% of propylene glycol showed an increase of a level to 46.5 mg/ml instead of values of around 20 mg/mL at pH 6 (shown in FIG. 2). Due to of the intensive depression freezing point, osmolality could not be tested.

### Example 3. Buffering

One finding from the previous Examples was that there is a range between pH 5 and 6 in which 60 mg/ml API can be dissolved. Strong precipitation of the free base only occurs above a pH of 5.9 according to the observation. Based on this observation, it was decided to investigate two compendial buffer systems (citrate and phosphate buffer) for their ability to hold 60 mg/ml of R-ketamine in solution and stabilize the formulation against pH shifts. Another change was that the pH adjustment was done with 0.15 N NaOH instead of 1 N NaOH in the following experiments. This change was underdone because there were concerns that the very alkaline microenvironment around the drops of sodium hydroxide solution would lead to the formation of flakes that were difficult to dissolve.

### Citrate Buffer

TABLE 8
Amounts of citric acid and sodium citrate to be mixed
Mix 0.1M Citric Acid with 0.1M Sodium Citrate in the proportions given below.

| | | | | | | | | | | | | | | | | | |
|---|---|---|---|---|---|---|---|---|---|---|---|---|---|---|---|---|---|
| pH | 3.0 | 3.2 | 3.4 | 3.6 | 3.8 | 4.0 | 4.2 | 4.4 | 4.6 | 4.8 | 5.0 | 5.2 | 5.4 | 5.6 | 5.8 | 6.0 | 6.2 |
| 0.1M Citric | 82.0 | 77.5 | 73.0 | 68.5 | 63.5 | 59.0 | 54.0 | 49.5 | 44.5 | 40.0 | 35.0 | 30.5 | 25.5 | 21.0 | 16.0 | 11.5 | 8.0 |
| Acid, mL | | | | | | | | | | | | | | | | | |
| 0.1M | 18.0 | 22.5 | 27.0 | 31.5 | 36.5 | 41.0 | 46.0 | 50.5 | 55.5 | 60.0 | 65.0 | 69.5 | 74.5 | 79.0 | 84.0 | 88.5 | 92.0 |
| Sodium | | | | | | | | | | | | | | | | | |
| Citrate, mL | | | | | | | | | | | | | | | | | |

### Experimental Procedure

The ingredients for a 20 mM buffer and the appropriate amount of R-ketamine HCl (60 mg/ml, equaling 52.2 mg/ml free base) were weighed together and diluted in water for injection. Subsequently, the pH was adjusted, e.g. to 5.2. After filling up to the target volume, the mixture was stirred for a defined time and a sample was obtained under filtration (0.22 μm). This sample was analyzed for content by HPLC and for tonicity by freezing point depression.

### Results

TABLE 9
Results 60 mg/ml R-ketamine in citrate buffer

| pH | Assay [mg/ml] | Tonicity [mOsmol/kg] |
|---|---|---|
| 5.2 | 46.2 | 534 |
| 5.4 | 46.1 | 540 |
| 5.6 | 46.4 | 546 |
| 5.8 | 47.2 | 561 |

The results for the citrate buffer (Table 9) show nearly constant content and tonicity in the range of pH 5.2-5.8. An observable trend towards higher contents at higher pH is within the range of the measurement accuracy of the method. Although no precipitation could be detected visually, only a content of about 46.5 mg/ml was found.

The series of experiments with 60 mg/ml R-ketamine was supplemented with a single study of 70 mg/ml (60.9 mg/ml free base) R-ketamine at pH 5.8 (table 10). Here, precipitation was observed after pH adjustment.

TABLE 10
Results of 70 mg/ml
R-Ketamine in citrate buffer

| pH | Assay [mg/ml] | Tonicity [mOsmol/kg] |
|---|---|---|
| 5.8 | 46.2 | 556 |

### Phosphate Buffer

Based on the previous results from the citrate buffer series experiment, the experiments with the phosphate buffer could be limited to pH 5.8. The nominal concentrations 60 and 70 mg/ml were investigated.

### Experimental Procedure

The solution was prepared and tested as described for the citrate buffer.

### Results

TABLE 11
Results R-Ketamine in phosphate buffer (pH 5.8)

| Nominal | Assay | Tonicity |
|---|---|---|
| Concentration (bulk) | [mg/ml free base] | [mOsmol/kg] |
| 60 mg/ml (52.2 mg/ | 51.2 | 476 |
| ml free base) | | |
| 70 mg/ml (60.9 mg/ | 59.7 | 545 |
| ml free base) | | |

After pH adjustment and filtration, both tests showed a slightly reduced active ingredient content compared to the original concentration, although no visible precipitation took place. Starting from a nominal 70 mg/ml R-ketamine (corresponding to 60.9 mg/ml free base), a content of 59.7 mg/ml was found in the filtered solution at pH 5.8. Accordingly, the targeted 60 mg/ml can be dissolved in water for injection under phosphate buffering at acceptable values for pH and tonicity. Also, the pH properties of the phosphate buffer are only slightly dependent on temperature, thus allowing end-sterilization of the formulation.

### Orientative Stability Examination

Subsequent storage at room temperature of the successfully tested phosphate-buffered formulation (pH 5.8) revealed limited shelf life, as a fine crystalline precipitate was observed after two weeks.

### Maleic Acid Buffering

Due to its ability to act as a buffer in the acidic to neutral range, a 0.02 M aqueous maleic acid solution was proposed as the base formulation. The aim was to investigate the dissolving capacity for R-ketamine in the concentration range of 60, 80 and 100 mg/ml at pH 5.5 and 5.7 (after the feasibility test with a pH of 5.8 had to be considered as failed) and the stability of the formulations in a study for four weeks.

Observed variables are visual appearance, assay and pH. For some of the 4-week samples, purity was also examined in addition to the assay. As a basis for comparison, the solution behaviour in water for injection (WFI) was observed at the same pH values.

Initial Tests with Maleic Acid Buffering

In an initial feasibility test, the dissolution behaviour of R-ketamine was investigated in the presence of maleic acid. Pure water was used as a comparison. In an aqueous solution of 60 mg/ml R-ketamine without buffering, a pH of 4.65 was obtained. If the same amount of R-ketamine was dissolved in a 0.02 M maleic acid buffer adjusted to pH 5.75, the pH decreased to a value of 5.38.

In the initial test, it was then possible to raise the pH to 5.8 with 0.15 M NaOH without precipitation of the active ingredient.

### Results (Water for Injection)

In addition to the above-mentioned experimental parameters, the possible influence of the alkali used for pH adjustment was also investigated (FIG. 4). KOH and NaOH were compared, and no difference was observed. It was possible to prepare clear solutions in the concentration range of 60-100 mg/ml API at both pH 5.5 and pH 5.7. The preparation of each sample was done separately.

### Four-Week Stability Assay

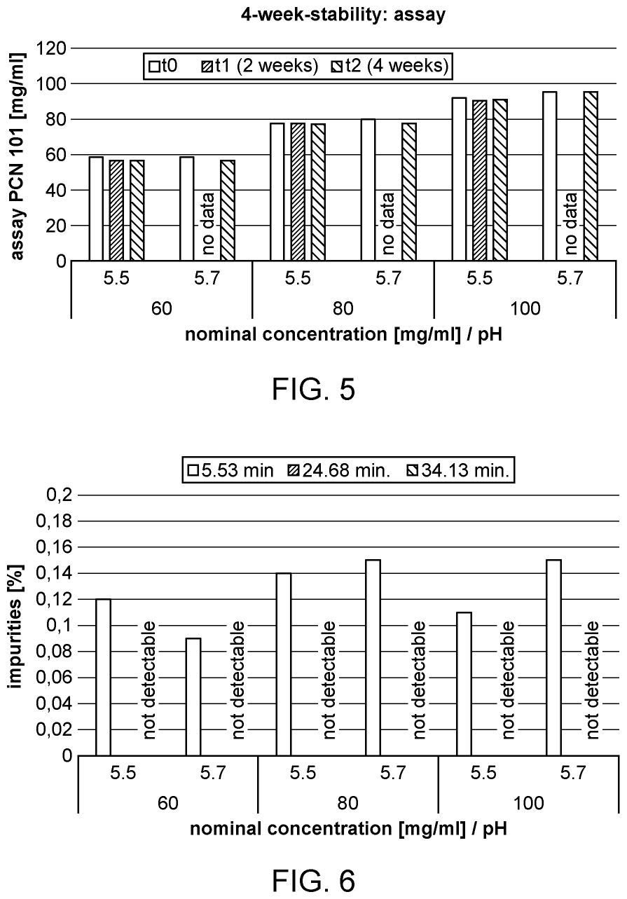

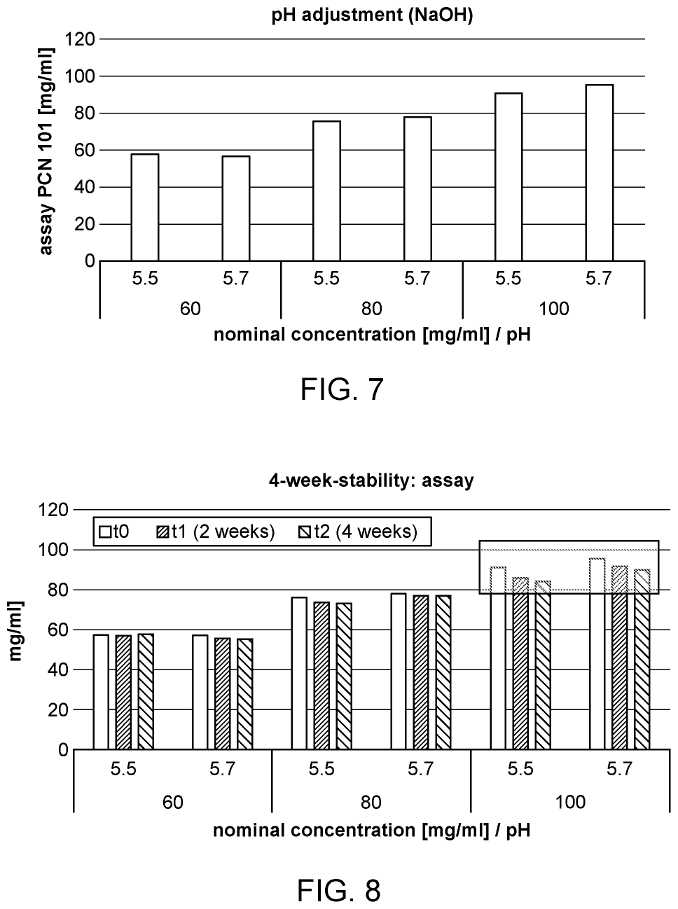

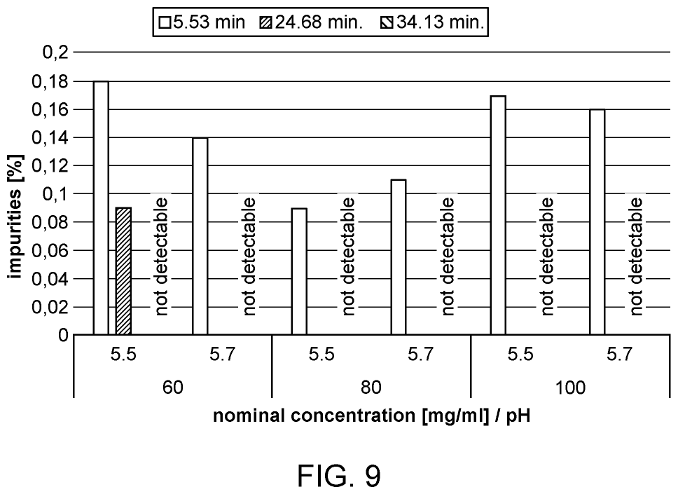

The stability study showed satisfactory stability after storage at room temperature for four weeks (FIG. 5). Visually, the samples presented as clear solutions without signs of precipitation.

### Four-Week Impurity Assay

The determination of impurities was performed on the four-week samples, and showed extensive stability.

### Four-Week Stability: pH

Regardless of the setting of pH 5.5 or 5.7, all samples showed a trend of decreasing pH over the duration of the study. On average, the change was 0.2 pH units, but in one case it also reached 0.4 units. Since the solution was unbuffered, these changes are plausible.

### Results (0.02 M Maleic Acid)

In addition to the above-mentioned experimental parameters, the possible influence of the alkali used for pH adjustment was also investigated. KOH and NaOH were compared, and no difference was observed.

### pH Dependence of the Solubility

It was possible to prepare clear solutions in the concentration range of 60-100 mg/ml API at both pH 5.5 and pH 5.7. The preparation of each sample was done separately.

### Four-Week Stability Assay

The stability study showed satisfactory stability after storage at room temperature for four weeks. Visually, the samples presented as clear solutions without signs of precipitation. The deviation from the nominal concentration was most pronounced at the highest dosage.

### Four-Week Impurity Assay

The determination of impurities was only performed on the four-week samples, and showed extensive stability.

### Four-Week Stability: pH

In the systems stabilized with maleic acid, the pH proved to be stable. In many cases, no change could be detected during storage.

### Summary

The solubility of R-ketamine is strongly pH dependent. In the acidic pH range, the active ingredient is readily soluble, while precipitation occurs at pH values greater than 5.7-5.9. A careful choice of formulation parameters allows the requirements of the application route to be reconciled with the dissolution behavior of the active ingredient. The way in which the pH is adjusted also has a major influence. By using low-concentration alkalis, the formation of highly alkaline microenvironments around the dropping point could be avoided. This prevents the formation of active ingredient flocs that are difficult to dissolve again.

### Example 4. R-ketamine Formulations in Maleate Buffer

R-ketamine formulations at various pH values were prepared in maleate buffer.

### Maleate Buffer Components:

Maleic acid, C₄H₄O₄, m.w. 116.07
NaOH, 1 N aqueous
Distilled H₂O
   The maleate buffer (100 mL at 20 mM) is prepared for example, by adding 0.232 g maleic acid, 2 mL NaOH, and by adding H₂O to 100 mL.
   R-Ketamine Formulation (40 mg/mL) at pH 5.0 in Maleate Buffer 20 mM and R-Ketamine Formulation (40 mg/mL) at pH 5.7 in Maleate Buffer 20 mM

The formulation is prepared by adding, for example, 40 mg/ml R-ketamine free base equivalent (46 mg/ml R-ketamine HCl salt) in 20 mM maleate buffer and the pH of the final formulation is adjusted using HCl or NaOH (below 0.2M or 0.15M) to equal a pH of 5.0 or a pH of 5.7.

R-Ketamine Formulation (80 mg/mL) at pH 5.0 in Maleate Buffer 20 mM and R-Ketamine Formulation (80 mg/mL) at pH 5.7 in Maleate Buffer 20 mM

The formulation is prepared by adding, for example, 80 mg/ml R-ketamine free base equivalent (92 mg/ml R-ketamine HCl salt) in 20 mM maleate buffer and the pH of the final formulation is adjusted using HCl or NaOH (below 0.2M or 0.15M) to equal a pH of 5.0 or a pH of 5.7.

### Example 5. R-ketamine Formulations in Fumarate Buffer

R-ketamine formulations at various pH values were prepared in fumarate buffer.

### Fumarate Buffer Components:

Fumaric acid, C₄H₄O₄, m.w. 116.07
NaOH, 1 N aqueous
Distilled H₂O
   The fumarate buffer (100 mL at 20 mM) is prepared for example, by adding 0.232 g fumaric acid, 2 mL NaOH, and by adding H₂O to 100 mL.
   R-Ketamine Formulation (40 mg/mL) at pH 5.0 in Fumarate Buffer 20 mM and R-Ketamine Formulation (40 mg/mL) at pH 5.7 in Fumarate Buffer 20 mM

The formulation is prepared by adding, for example, 40 mg/ml R-ketamine free base equivalent (46 mg/ml R-ketamine HCl salt) in 20 mM fumarate buffer and the pH of the final formulation is adjusted using HCl or NaOH (below 0.2M or 0.15M) to equal a pH of 5.0 or a pH of 5.7.

R-Ketamine Formulation (80 mg/mL) at pH 5.0 in Fumarate Buffer 20 mM and R-Ketamine Formulation (80 mg/mL) at pH 5.7 in Fumarate Buffer 20 mM

The formulation is prepared by adding, for example, 80 mg/ml R-ketamine free base equivalent (92 mg/ml R-ketamine HCl salt) in 20 mM fumarate buffer and the pH of the final formulation is adjusted using HCl or NaOH (below 0.2M or 0.15M) to equal a pH of 5.0 or a pH of 5.7.

### Example 6. R-Ketamine Formulations in Citrate Buffer

R-ketamine formulations at various pH values were prepared in citrate buffer, and the citrate buffer was prepared accordingly.

### Prepare 1 Liter Citrate Buffer 20 mM pH 5

| Component | Amount | Concentration |
|---|---|---|
| Sodium Citrate dihydrate | 3.42 g | 0.0116M |
| (mw: 294.10 g/mol) | | |
| Citric Acid (mw: 192.12 g/mol) | 1.608 g | 0.0084M |

1. Prepare 800 mL of distilled water in a suitable container.
2. Add 3.42 g of sodium citrate dihydrate to the solution.
3. Add 1.608 g of citric acid to the solution.
4. Adjust solution to final desired pH using HCl or NaOH
5. Add distilled water until volume is 1 L.

### Prepare 1 Liter Citrate Buffer at pH 5.7

| Component | Amount | | Concentration |
|---|---|---|---|
| Sodium Citrate dihydrate | 4.424 | g | 0.015M |
| (mw: 294.10 g/mol) | | | |
| Citric Acid | 953 | mg | 0.005M |
| (mw: 192.12 g/mol) | | | |

1. Prepare 800 mL of distilled water in a suitable container.
2. Add 4.424 g of sodium citrate dihydrate to the solution.
3. Add 0.953 g of citric acid to the solution.
4. Adjust solution to final desired pH using HCl or NaOH
5. Add distilled water until volume is 1 L.
   R-Ketamine Formulation (40 mg/mL) at pH 5.0 in Citrate Buffer 20 mM and R-Ketamine Formulation (40 mg/mL) at pH 5.7 in Citrate Buffer 20 mM

The formulation is prepared by adding, for example, 40 mg/ml R-ketamine free base equivalent (46 mg/ml R-ketamine HCl salt) in 20 mM citrate buffer and the pH of the final formulation is adjusted using HCl or NaOH (below 0.2M or 0.15M) to equal a pH of 5.0 or a pH of 5.7.

R-Ketamine Formulation (80 mg/mL) at pH 5.0 in Citrate Buffer 20 mM and R-Ketamine Formulation (80 mg/mL) at pH 5.7 in Citrate Buffer 20 mM

The formulation is prepared by adding, for example, 80 mg/ml R-ketamine free base equivalent (92 mg/ml R-ketamine HCl salt) in 20 mM citrate buffer and the pH of the final formulation is adjusted using HCl or NaOH (below 0.2M or 0.15M) to equal a pH of 5.0 or a pH of 5.7.

### Example 7. R-Ketamine Formulations in Succinate Buffer

R-ketamine formulations at various pH values were prepared in succinate buffer.

### Succinate Buffer Components:

Succinic acid, C₄H₄O₄, m.w. 118.09
NaOH, 1 N aqueous
Distilled H₂O
   The fumarate buffer (100 mL at 20 mM) is prepared for example, by adding 0.236 g succinic acid, 2 mL NaOH, and by adding H₂O to 100 mL.
   R-Ketamine Formulation (40 mg/mL) at pH 5.0 in Succinate Buffer 20 mM and R-Ketamine Formulation (40 mg/mL) at pH 5.7 in Succinate Buffer 20 mM

The formulation is prepared by adding, for example, 40 mg/ml R-ketamine free base equivalent (46 mg/ml R-ketamine HCl salt) in 20 mM succinate buffer and the pH of the final formulation is adjusted using HCl or NaOH (below 0.2M or 0.15M) to equal a pH of 5.0 or a pH of 5.7.

R-Ketamine Formulation (80 mg/mL) at pH 5.0 in Succinate Buffer 20 mM and R-Ketamine Formulation (80 mg/mL) at pH 5.7 in Succinate Buffer 20 mM

The formulation is prepared by adding, for example, 80 mg/ml R-ketamine free base equivalent (92 mg/ml R-ketamine HCl salt) in 20 mM succinate buffer and the pH of the final formulation is adjusted using HCl or NaOH (below 0.2M or 0.15M) to equal a pH of 5.0 or a pH of 5.7.

### Example 8. R-ketamine Formulations in Tartrate Buffer

R-ketamine formulations at various pH values were prepared in tartrate buffer.

### Tartrate Buffer Components:

Tartaric acid, C₄H₆O₆, m.w. 150.09
Sodium tartrate dihydrate, m.w. 230.08
NaOH, 1 N aqueous
Distilled H₂O

The tartrate buffer (1 L at 5 mM) is prepared for example, by adding 0.75 g tartaric acid, 2 mL NaOH, and by adding H₂O to 1 L. The tartrate buffer (1 L at 15 mM) is prepared for example, by adding 3.452 g sodium tartrate dihydrate, 2 mL dil. acid, and by adding H₂O to 1 L. The tartrate buffer (1 L at 20 mM) is prepared for example, by adding 4.6 g sodium tartrate dihydrate, 2 mL dil. acid, and by adding H₂O to 1 L.

R-Ketamine Formulation (40 mg/mL) at pH 5.0 in Tartrate Buffer 20 mM and R-Ketamine Formulation (40 mg/mL) at pH 5.7 in Tartrate Buffer 20 mM

The formulation is prepared by adding, for example, 40 mg/ml R-ketamine free base equivalent (46 mg/ml R-ketamine HCl salt) in 20 mM tartrate buffer and the pH of the final formulation is adjusted using HCl or NaOH (below 0.2M or 0.15M) to equal a pH of 5.0 or a pH of 5.7.

R-Ketamine Formulation (80 mg/mL) at pH 5.0 in Tartrate Buffer 20 mM and R-Ketamine Formulation (80 mg/mL) at pH 5.7 in Tartrate Buffer 20 mM

The formulation is prepared by adding, for example, 80 mg/ml R-ketamine free base equivalent (92 mg/ml R-ketamine HCl salt) in 20 mM tartrate buffer and the pH of the final formulation is adjusted using HCl or NaOH (below 0.2M or 0.15M) to equal a pH of 5.0 or a pH of 5.7.

### EQUIVALENTS

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

What is claimed is:

1. A formulation for subcutaneous administration comprising R-ketamine or a pharmaceutically acceptable salt thereof.

2. The formulation of claim 1, substantially free of S-ketamine and/or a pharmaceutically acceptable salt thereof.

3. The formulation of claim 1 or 2, wherein the R-ketamine is present at a concentration between about 60 mg/mL and about 150 mg/mL.

4. The formulation of any one of claims 1-3, wherein the R-ketamine is present at a concentration between about 70 mg/mL and about 120 mg/mL.

5. The formulation of any one of claims 1-4, wherein the R-ketamine is present at a concentration between about 70 mg/mL and about 100 mg/mL.

6. The formulation of any one of claims 1-5, wherein the R-ketamine is present at a concentration between about 75 mg/mL and about 100 mg/mL.

7. The formulation of any one of claims 1-3, wherein the R-ketamine is present at a concentration of about 60 mg/mL, 65 mg/mL, 70 mg/mL, 75 mg/mL, 80 mg/mL, 85 mg/mL, 90 mg/mL, about 95 mg/mL, about 100 mg/mL, about 105 mg/mL or about 110 mg/mL.

8. The formulation of any one of claims 1-7, wherein the pH of the formulation is about 6.0 or less.

9. The formulation of any one of claims 1-8, wherein the pH of the formulation is about 5.0, about 5.1, about 5.2, about 5.3, about 5.4, about 5.5, about 5.6, about 5.7, about 5.8, about 5.9, or about 6.0.

10. The formulation of any one of claims 1-9, wherein the pH of the formulation is between about 5.70 and about 5.80.

11. The formulation of any one of claims 1-10, wherein the subcutaneous formulation is below a pH of about 5.8.

12. The formulation of any one of claims 1-11, wherein the subcutaneous formulation is at a pH of about 5.8.

13. The formulation of any one of claims 1-11, wherein the subcutaneous formulation is at a pH of about 5.75.

14. The formulation of any one of claims 1-13, wherein the subcutaneous formulation further comprises a buffer.

15. The formulation of any one of claims 1-14, wherein the subcutaneous formulation further comprises a buffer selected from one or more of a succinate buffer, tartrate buffer, phosphate buffer, a maleate buffer, a fumarate buffer, a citrate buffer and an acetate buffer.

16. The formulation of any one of claims 1-15, wherein the formulation comprises R-ketamine HCl or R-ketamine free base.

17. An R-ketamine formulation for subcutaneous injection comprising:
a) R-ketamine between about 90 mg/mL and about 110 mg/mL; and
b) a buffering agent;
wherein the pH of the formulation is 5.8 or less, and wherein the formulation is substantially free of S-ketamine.

18. The formulation of claim 17, wherein the R-ketamine is present at a concentration of about 90 mg/mL, about 95 mg/mL, about 100 mg/mL, about 105 mg/mL or about 110 mg/mL.

19. The formulation of claim 17 or 18, wherein the pH of the formulation is about 5.8.

20. The formulation of claim 17 or 18, wherein the pH of the formulation is about 5.75.

21. The formulation of claim 17 or 18, wherein the pH of the formulation is about 5.7.

22. The formulation of any one of claims 17-21, wherein the buffering agent is selected from one or more of a phosphate buffer, a maleate buffer, a fumarate buffer, a citrate buffer and an acetate buffer.

23. The formulation of any one of claims 17-22, wherein the buffering agent is a maleate buffer.

24. The formulation of any one of claims 17-22, wherein the buffering agent is a fumarate buffer.

25. The formulation of any one of claims 17-22, wherein the buffering agent is a citrate buffer.

26. The formulation of any one of claims 17-22, wherein the buffering agent is an acetate buffer.

27. A process for preparing the formulation according to any one of claims 1-26, comprising adjusting the pH using a dilute acid or dilute base.

28. The process of claim 27, comprising adjusting the pH using a dilute acid or dilute base with concentration less than 0.5N, less than 0.6N, less than 0.7N, less than 0.8N, less than 0.9N, or less than about 1N.

29. The process of claim 27 or 28, comprising adjusting the pH using either a dilute KOH solution or a dilute NaOH solution.

30. The process of claim 27, comprising adjusting the pH using a basic solution of about 1N.

31. The process of claim 27, comprising adjusting the pH using either a KOH solution with a concentration of 1N or less or a NaOH solution with a concentration of 1N or less.

32. The process of claim 27, comprising adjusting the pH using a 1N NaOH solution or a 1N KOH solution.

33. The process of claim 27, comprising adjusting the pH using a 0.5N NaOH solution or a 0.5N KOH solution.

34. The process of any one of claims 27-33, wherein the process facilitates increased solubility of the R-ketamine at a pH of between about 5.6 and about 6.0 or between about 5.6 and about 5.8.

35. The process of claims 27-33, wherein the process facilitates increased solubility of the R-ketamine at a pH of about 6.0, about 5.9, about 5.8, or about 5.75, or about 5.7, or about 5.6.

36. An injection device comprising a needle suitable for subcutaneous administration and the formulation according to any one of claims 1-26.

37. A kit comprising the formulation according to any one of claims 1-26 and instructions for use thereof.

38. A method of treating a disease or condition in a patient in need thereof, comprising administering to the patient the formulation according to any one of claims 1-26.

39. The method of claim 38, wherein the formulation is administered via subcutaneous injection.

40. Use of the formulation of any one of claims 1-26 for the treatment of a disease or condition.
